(19) United States
(12) Patent Application Publication
(10) Pub. No.: US 2020/0232995 A1
(43) Pub. Date: Jul. 23, 2020

(54) MEASUREMENT OF PROTEIN MOLECULAR FLUX RATES BY QUANTIFYING ISOTOPOLOGUE ABUNDANCES IN IMMONIUM ION USING HIGH RESOLUTION MASS SPECTROMETRY

(71) Applicant: KineMed, Inc., Piedmont, CA (US)

(72) Inventors: Marc K. HELLERSTEIN, Kensington, CA (US); Thomas E. ANGEL, Trappe, PA (US); Alexander A. MAKAROV, Bremen (DE)

(73) Assignee: KineMed, Inc., Piedmont, CA (US)

(21) Appl. No.: 15/758,268

(22) PCT Filed: Sep. 7, 2016
(86) PCT No.: PCT/US2016/050583

§ 371 Date: Mar. 7, 2018

Related U.S. Application Data
(60) Provisional application No. 62/215,657, filed on Sep. 8, 2015.

(51) Int. Cl. G01N 33/68; G16B 25/00; G16B 40/10; G01N 33/58

(52) U.S. Cl. CPC G01N 33/6848; G16B 25/00; G01N 2458/15; G01N 33/58; G01N 2560/00; G16B 40/10

(57) ABSTRACT

Provided herein are methods for measuring a molecular flux rate proteins and polypeptides based on analysis of isotopologue abundances of immonium ion fragments within a mass isotopomer using a high resolution mass spectrometric measurement. Such methods may be used, inter alia, to calculate a fraction of newly synthesized protein or polypeptide molecules of interest, a replacement rate of target molecules of interest, and/or a rate of breakdown or degradation of target molecules of interest, e.g., based on isotopologue relative abundances.

### FIELD

The present disclosure relates generally to methods for measuring a molecular flux rate proteins or polypeptides based on analysis of isotopologue abundances within mass isotopomers of immonium ion fragments, e.g., using a high resolution mass spectrometric measurement.

### BACKGROUND

Measurements of molecular flux rates, or kinetics, of processes that are causal in disease represent the next generation of medical biomarkers (Hellerstein, M. K. (2003) Annu. Rev. Nutr. 23:379-402; Turner, S. M. and Hellerstein, M. K. (2005) Curr. Opin. Drug Discov. Devel. 8:115-126; Hellerstein, M. K. (2008) Metab. Eng. 10:1-9; and Hellerstein, M. K. (2008) J. Pharmacol. Exp. Ther. 325:1-9). More efficient drug development, personalized medicine, medical diagnostics and wellness monitoring would all be considerably advanced by the availability of objective metrics that revealed the rates at which pathogenic molecular processes are occurring in living humans. Measurement of molecular kinetics or flux rates in vivo generally requires perturbing a system by introducing a tag, or isotopic label, and measuring its rate of flow into molecules of interest. The reason for this is that introduction of a label results in an asymmetry in the dimension of time (i.e., label was not present, then it was), so that time-dependent changes, or kinetic transients, can be measured, and the rates of observable processes can be calculated.

Over the past three decades, kinetic measurements in living systems have evolved from predominantly using radioactive isotopic tracers to the use of non-radioactive, stable isotopic tracers, particularly heavy water (²H₂O). The latter are generally measured by use of mass spectrometry (MS) or nuclear magnetic resonance. This transition was initially largely motivated by the safety and toxicity problems associated with and caused by radioisotopes, which are avoided by use of stable isotopes (Wolfe, R. R. and Chinkes, D. L. (2004) Isotope Tracers in Metabolic Research: Principles and Practice of Kinetic Analysis, 2^{nd} ed. Wiley). As mass spectrometers have dramatically improved, however, it has become apparent that stable isotope-MS methods provide a number of unique capabilities for molecular kinetic measurements that are not possible with radioisotope approaches (see, e.g., Hellerstein, M. K. and Neese, R. A. (1992) Am. J. Physiol. 263: E988-1001; and Hellerstein, M. K. and Neese, R. A. (1999) Am. J. Physiol. 276: E1146-1170). Two of the basic advantages provided by stable isotope-MS approaches are the capacity to measure different patterns of isotope labeling within individual molecules and the ability to detect and analyze molecular fragment ions.

Radioisotopic measurements—e.g., through liquid scintillation counting or Geiger counting—measure the total amount of label in a population of molecules, with the result typically expressed as disintegrations per minute (dpm)/mole (Wolfe, R. R. and Chinkes, D. L. (2004) Isotope Tracers in Metabolic Research: Principles and Practice of Kinetic Analysis, 2^{nd} ed. Wiley). The pattern or variability of labeling among individual molecules in the population, however, cannot be measured by this approach because individual molecules are not isolated, identified or detected.

In contrast, MS analysis is capable of detecting the relative abundances of different molecular species, or isotopic isomers that are present in a sample. One category of isotopic isomers is mass isotopomers, which are defined as isotopic isomers that differ in elemental isotope composition and nominal mass—e.g., single-labeled vs. double-labeled species of a molecule. Many applications of mass isotopomer analysis have been discovered (Hellerstein, M. K. and Neese, R. A. (1992) Am. J. Physiol. 263: E988-1001; Hellerstein, M. K. and Neese, R. A. (1999) Am. J. Physiol. 276: E1146-1170; Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286: E577-588), in particular through use of “mass isotopomer distribution analysis” (MIDA; see, e.g., U.S. Pat. No. 5,338,686). MIDA is a method that includes quantifying changes in the relative abundances of different mass isotopomers of a molecule (e.g., the relative proportions of unlabeled, single-labeled, double-labeled, etc. molecular species), compared to natural abundances of the mass isotopomers, and then inferring from this internal labeling pattern the isotopic content of the true biosynthetic precursor pool at the intracellular site of biosynthesis, through application of equations based on the binomial or multinomial distribution. The pattern of excess abundances or enrichments of each mass, termed (EM1, EM2, EM3, etc.) thereby reveals the fraction of labeled atoms in the biosynthetic precursor pool (Hellerstein, M. K. and Neese, R. A. (1992) Am. J. Physiol. 263: E988-1001; and Hellerstein, M. K. and Neese, R. A. (1999) Am. J. Physiol. 276: E1146-1170). Knowledge of the label enrichment of the true biosynthetic precursor pool, e.g., calculated in this manner, allows essentially all parameters related to molecular fluxes of the molecule to be calculated with rigorous accuracy. MIDA has been called “the equation for biosynthesis.” (Hellerstein, M. K. and Neese, R. A. (1992) Am. J. Physiol. 263: E988-1001; and Hellerstein, M. K. and Neese, R. A. (1999) Am. J. Physiol. 276: E1146-1170). The capacity to measure mass isotopomer abundances (MIAs) and mass isotopomer distributions (MIDs) has been applied to kinetic analysis of lipids (Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286: E577-588), intermediary metabolites (Neese, R. A. et al. (1995) J. Biol. Chem. 270:14452-14466; Hellerstein, M. K. et al. (1997) Am. J. Physiol. 272: E163-172; Hellerstein, M. K. et al. (1997) J. Clin. Invest. 100:1305-1309; and Louie, K. B. et al. (2013) Sci. Rep. 3:1656), proteins (Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814) and cells (Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057). Of note, all of these molecular flux rates can be measured by use of ²H₂O labeling.

Broad application of molecular kinetic measurements for routine clinical medical management and drug development would have a potentially transformative effect on these areas of biomedicine. The power of kinetic measurements to capture the activity of disease-driving processes could radically advance personalized medicine and the efficiency of testing drug candidates in FDA trials (Messmer, B. T. et al. (2005) J. Clin. Invest. 115:755-764; and Decaris, M. L. et al. (2015) PLoS One 10:e0123311).

There are major technical limitations and constraints, however that continue to prevent broad or routine applications of mass spectrometric kinetic analysis in clinical medicine and drug development. The most important of these limitations is the inability of the current generation of mass spectrometers to achieve sufficiently accurate and precise measurements of MIAs and MIDs in organic biomolecules for routine medical applications. Mass spectrometers including quadrupole, ion trap, time-of-flight (ToF), magnetic sector, and Fourier transform-ion cyclotron resonance (FT-ICR) including Orbitrap mass analyzers, to name a few, along with hybrid instrument modalities, have not had sufficient analytic precision and accuracy for MIAs and MIDs to allow routine application of molecular kinetic methods in clinical medicine or in drug development settings, particularly with the relatively low isotopic perturbations induced by ²H₂O labeling in the clinical setting, where dose and duration of ²H₂O administration are limited by safety and practical factors.

The problems presented by these limitations is illustrated by considering the following example. A quadrupole GC/MS when performing optimally can routinely achieve reproducibility for MIA quantitation of ±˜0.1-0.2% (±0.001-0.002 fractional abundance) for each mass isotopomer in an isotope envelope of a typical small molecular ion (e.g., m/z<800). A different type of instrument, for example a Q-ToF, for larger molecules when operating optimally can achieve analytic accuracy and reproducibility of 0.3-0.5% (see e.g., Wolfe, R. R. and Chinkes, D. L. (2004) Isotope Tracers in Metabolic Research: Principles and Practice of Kinetic Analysis, 2^{nd} ed. Wiley; Hellerstein, M. K. and Neese, R. A. (1992) Am. J. Physiol. 263: E988-1001; Hellerstein, M. K. and Neese, R. A. (1999) Am. J. Physiol. 276: E1146-1170; Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814). This analytic performance translates into the following heavy water (²H₂O) labeling protocol requirements for human subjects. A convenient simulation with a fairly typical ˜1.0% enrichment of ²H₂O in body water in humans is shown in Table 1, with the analyzed molecule being 600 daltons and containing 6 potential deuterium labeling sites (k refers to the turnover rate constant). EM1 refers to the change (enrichment) in relative abundance of the M1-mass isotopomer of the molecule, after correcting for natural abundance or baseline of the M1-mass isotopomer of the molecule.

TABLE 1
Label Incorporation Kinetics in Relation to Analytic Detection
Limits (for a molecule of ~600 daltons with 6 potential
labeling sites at body water enrichment of 1.0%).

| k | % Labeled EM1 | % Labeled EM1 | % Labeled EM1 |
|---|---|---|---|
| (%/day) | (day 1) | (day 5) | (day 10) |
| 1 | 0.03 | 0.15 | 0.30 |
| 2 | 0.06 | 0.30 | 0.60 |
| 5 | 0.15 | 0.75 | 1.50 |

As shown in Table 1, detection of sufficient label incorporation into a molecule of mass ˜600 Da with a turnover rate between 1%-5% per day (i.e., a half-life of from 2 weeks to ˜2 months) requires at least several days of label exposure at 1% body water enrichment to achieve label incorporation in the targeted molecule that is adequate in terms of signal to noise ratio for reliable quantitation. Typically, this requires enrichment values that are several times the limit of accurate detection of 0.1-0.5% EM1 and therefore enrichment values of at least 0.50-1.0%. Clearly, there is a need for analytic improvements that can allow the use of a clinical labeling protocol that is substantially less burdensome and less expensive (e.g., a protocol that involves giving a few oral doses of heavy water, ²H₂O, over 1-2 days), and such improvements would represent a major practical advance for applying ²H₂O labeling methods in medical diagnostics and drug development.

The most powerful class of commercial mass spectrometers in terms of mass resolution, mass measurement accuracy, sensitivity, and analytical breadth and depth for detection and identification of molecules are the FT-ICR instruments, such as the Orbitrap (Scheltema, R. A. et al. (2014) Mol. Cell Proteomics 13:3698-3708). However, measurement of MIAs and MIDs with FT-ICR has consistently been found to be even less accurate and reproducible than is routinely achieved by much less powerful instruments, such as ToF or quadrupole instruments (see e.g., Erve, J. C. et al. (2009) J. Am. Soc. Mass Spectrom. 20:2058-2069; and Mathur, R. and O'Connor, P. B. (2009) Rapid Commun. Mass. Spectrom. 23:523-529). The reasons for this poor quantitative performance by FT-ICR instruments is not understood, and a number of possible reasons have been proposed. One potential reason is the mass-dependent differential decay rates of transients for ions within the trap (see e.g., Erve, J. C. et al. (2009) J. Am. Soc. Mass Spectrom. 20:2058-2069; and Mathur, R. and O'Connor, P. B. (2009) Rapid Commun. Mass. Spectrom. 23:523-529). This phenomenon relates to different behavior of ions in the trap that is a function of their mass, as well as on their intensity or abundance. Differential decay rates of ions result in biases in the proportion of ions that remain in the trap and are detected. The result of these biases is the inaccurate measurement of true isotope ratios for the ions that entered the trap. It has been observed that the greater the difference in mass or in abundance, the greater the potential biasing effect on measured relative abundances (see e.g., Perry, R. H. et al. (2008) Mass. Spectrom. Rev. 27:661-699; and Scheltema, R. A. et al. (2014) Mol. Cell Proteomics 13:3698-3708). Another potential explanation is the contamination of mass isotopomers with ions derived from the biologic matrix in which the target molecular was measured. Random or systematic background “multiplexing” of contaminants are especially problematic for molecules that are of low abundance in a biologic matrix, which is often the case and is indeed an advantage of sensitive mass spectrometers such as FT-ICR instruments. In addition, an explanation for the insufficient analytic sensitivity of mass spectrometers in general, including FT-ICR instruments, for quantitation of very small changes in MIAs and MIDs after low doses of isotopic precursors such as ²H₂O relates to the issue of relative change in analytic signal. For mass isotopomers, the natural fractional abundance of the M0, M1, M2, etc. mass isotopomers is typically a much larger number than the change or perturbation in mass isotopomer fractional abundance after introduction of small amounts of ²H after metabolic labeling with a low dose of ²H₂O. The signal-to-noise characteristics of such measurements are not optimal for reproducible measurements.

Taken together, these many limitations have prevented the broad application of these exquisitely sensitive and powerful instruments for development of kinetic biomarkers in clinical medicine and biomedical research. Accordingly, new methods are needed that can take advantage of the high mass resolution, mass measurement accuracy, and sensitivity of high resolution mass spectrometers. These methods should provide improved accuracy and precision for MIA and MID measurements as compared to current instruments, and be less susceptible to interferences from contaminating ions in complex analytic matrices. Such new methods would be useful and broadly applicable across biology, medicine, and clinical practice.

### SUMMARY

A central analytic requirement for measuring molecular flux rates is accurate and precise measurements of changes in relative abundances of the labeled species of targeted molecules of interest after introduction of a labeled metabolic precursor. The imprecision and inaccuracy of current techniques for quantifying relative abundances of different labeled species (specifically, mass isotopomers) in targeted molecules is a major problem currently holding back broad application molecular flux rate measurements in medical practice and biomedical research, particularly for use of the most universal and clinically useful heavy water labeling approach, which generates relatively modest changes in mass isotopomer abundances.

The present disclosure provides methods that solve the problem of measuring molecular flux rates in humans and pre-clinical models. The disclosed methods have several advantageous features including: (1) use of the high mass resolution, high mass measurement accuracy, and high sensitivity of FT-ICR instruments; (2) decreased susceptibility to interference from contaminating ions from complex analytic matrices; and (3) increased accuracy and precision compared to current FT-ICR measurement methods of MIAs and MIDs achieved by (i) reducing the impact of differences in decay rates of transients due to different mass-to-charge ratios and different intensities of ions; (ii) increasing the relative change in abundance ratios of the labeled species (masses) that are analyzed in molecules after low-dose or short-term labeling, compared to baseline molecules before low-dose or short-term labeling, and (iii) maintaining the relative abundances of comparable signal intensities in the measured peaks in target molecules, to reduce the potential analytic impact of non-linearities of detector quantitation or differences in ion counting statistics. These advantageous features allow for the potential broad application of the methods across biology and medicine, particularly for use of low-dose or short-term ²H₂O labeling protocols in the clinical setting. The methods disclosed herein provides these advantages by targeting a unique feature of stable isotope labeling that has not previously been used for the measurement of molecular flux rates. The methods disclosed herein utilize the measurement of the relative abundances of isotopologues within mass isotopomers of a molecule, which differ in mass by millidaltons. These methods use high mass-resolution mass spectrometry (e.g., use of FT-ICR instruments) to measure changes in relative abundances of isotopologues and thereby provide analytic advantages, particularly for ²H₂O labeling approaches. These methods are particularly advantageous when changes in mass isotopomer abundances are relatively modest, as occurs for low dose or brief duration ²H₂O labeling protocols that are optimal for broad medical diagnostic applications of molecular flux rate measurements. Accordingly, provided herein, inter alia, are methods for metabolically labeling and analyzing relative abundances of isotopologues in selected mass isotopomers by use of high mass-resolution mass spectrometers, as well as methods for calculating molecular flux rates of molecules by this approach.

In certain aspects, the present disclosure relates to a method for measuring a molecular flux rate or fractional synthesis rate of a protein or polypeptide based on analysis of isotopologue abundance within a mass isotopomer from an immonium ion fragment, comprising: (a) administering a stable isotope-labeled precursor molecule to a subject for a period of time sufficient for said stable isotope-labeled precursor molecule to enter into a biosynthetic precursor pool and label one or more protein or polypeptide molecules of interest to produce one or more stable isotope-labeled target molecules of interest; (b) obtaining from the subject a biological sample comprising the one or more stable isotope-labeled protein or polypeptide molecules of interest; (c) enriching or isolating the one or more stable isotope-labeled protein or polypeptide molecules of interest from said biological sample; (d) performing a high resolution mass spectrometric measurement of a relative abundance of a first isotopologue from said enriched or isolated one or more stable isotope-labeled protein or polypeptide molecules of interest, wherein the relative abundance of the first isotopologue is a ratio of abundance of the first isotopologue to a sum of (i) the abundance of the first isotopologue and (ii) an abundance of a second isotopologue from said enriched or isolated one or more stable isotope-labeled protein or polypeptide molecules of interest, in a preferred embodiment wherein the first and the second isotopologues have different exact masses and are part of the same mass isotopomer of an immonium ion fragment; (e) comparing the relative abundance of the first isotopologue to a control relative abundance of the first isotopologue, wherein the control relative abundance is a ratio of abundance of the first isotopologue from the one or more protein or polypeptide molecules of interest before or without administration of the stable isotope-labeled precursor molecule to a sum of (i) the abundance of the first isotopologue before or without administration of the stable isotope-labeled precursor molecule and (ii) an abundance of the second isotopologue from the one or more protein or polypeptide molecules of interest without administration of the stable isotope-labeled precursor molecule; and (f) calculating a fraction of newly synthesized protein or polypeptide molecules of interest based on the comparison of the relative abundance of the first isotopologue and the control relative abundance of the first isotopologue. In certain embodiments, the method further comprises calculating a replacement rate of the protein or polypeptide molecules of interest based on the calculated fraction of newly synthesized protein or polypeptide molecules of interest.

In some embodiments of the method for measuring a molecular flux rate or fractional synthesis rate of a protein or polypeptide based on analysis of isotopologue abundance within a mass isotopomer from an immonium ion fragment, as disclosed herein, the method is carried out wherein the stable isotope-labeled precursor molecule is ²H₂O. In some embodiments, the method is carried out wherein the first isotopologue is a ²H-isotopologue, and wherein the second isotopologue is a ¹³C isotopologue. In some embodiments, the method is carried out wherein the stable isotope-labeled precursor molecule is selected from the group consisting of a ¹⁵N-labeled amino acid, a ¹⁵N-labeled polypeptide, and a ¹⁵N-labeled inorganic nitrogenous compound. In some embodiments, the method is carried out wherein the stable isotope-labeled precursor molecule is selected from the group consisting of a ¹³C-labeled amino acid, a ¹³C-labeled polypeptide, a ¹³C-labeled organic metabolite, and a ¹³C-labeled inorganic carbon compound. In some embodiments, the method is carried out wherein the stable isotope-labeled precursor molecule is ¹⁷O-labeled H₂O or ¹⁸O-labeled H₂O.

In some embodiments of the method for measuring a molecular flux rate or fractional synthesis rate of a protein or polypeptide based on analysis of isotopologue abundance within a mass isotopomer from an immonium ion fragment, the method is carried out, wherein the mass isotopomer is an M1-mass isotopomer.

In some embodiments of the method for measuring a molecular flux rate or fractional synthesis rate of a protein or polypeptide based on analysis of isotopologue abundance within a mass isotopomer from an immonium ion fragment, the method is carried out wherein the production of molecular ions and isolation of fragment ions occurs on a high-resolution mass spectrometer operating at such resolving power that isotope label can be resolved on at least one fragment of the molecule, wherein the abundance of isotope label in the original molecular ions is established on the basis of abundance of the isotope label in said fragment as well as on probability of the particular isotope label to propagate from the molecular ion to the fragment ion.

In some embodiments of the method for measuring a molecular flux rate or fractional synthesis rate of a protein or polypeptide based on analysis of isotopologue abundance within a mass isotopomer from an immonium ion fragment, the method further comprises obtaining from the subject at least a second biological sample comprising the one or more stable isotope-labeled protein or polypeptide molecules of interest, wherein the first and second biological samples are obtained at different times, and wherein calculating the fraction of newly synthesized protein or polypeptide molecules of interest comprises calculating a fraction of protein or polypeptide molecules of interest synthesized before obtaining the first biological sample and a fraction of protein or polypeptide molecules of interest synthesized before obtaining the second biological sample.

In some embodiments of the method for measuring a molecular flux rate or fractional synthesis rate of a protein or polypeptide based on analysis of isotopologue abundance within a mass isotopomer from an immonium ion fragment, the method is carried out wherein the high resolution mass spectrometric measurement is performed using a high resolution mass spectrometer capable of quantifying isotopologues that differ in mass by nine or fewer millidaltons. In some embodiments, the method is carried out wherein the high resolution mass spectrometric measurement is performed using a high resolution mass spectrometer capable of quantifying isotopologues that differ in mass by three or fewer millidaltons. In some embodiments, the method is carried out wherein the high resolution mass spectrometer is an FT-ICR mass spectrometer.

In some embodiments of the method for measuring a molecular flux rate or fractional synthesis rate of a protein or polypeptide based on analysis of isotopologue abundance within a mass isotopomer from an immonium ion fragment, the method is carried out wherein the relative abundances of the first isotopologue and the second isotopologue are of comparable peak heights or signal intensities (e.g., within 50% of each other). In some embodiments of the method the calculated fraction of newly synthesized protein or polypeptide molecules of interest, the replacement rate of the protein or polypeptide molecules of interest, the rate of breakdown or degradation of the protein or polypeptide molecules of interest, or any combination thereof is used in the diagnosis, management, or treatment selection of a human or veterinary patient.

In another embodiment, the present disclosure provides a method of measuring a molecular flux rate or fractional synthesis rate of a protein or polypeptide comprising: (a) performing a high resolution mass spectrometric measurement of a stable-isotope labeled precursor exposed sample and a control sample, wherein each sample comprises the protein or polypeptide; (b) determining from the measurement of each sample the relative abundance of a first isotopologue to a second isotopologue, wherein the first and second isotopologues are part of the same mass isotopomer of an immonium ion fragment derived from the protein or polypeptide; and (c) calculating the molecular flux rate or fractional synthesis rate of the protein or polypeptide based on the determined relative abundances of the first isotopologue to the second isotopologue for the exposed and control samples. In some embodiments, this method is carried out wherein step (b) is repeated to determine relative abundances for a first isotopologue to a second isotopologue in a plurality of different immonium ion fragments derived from the protein or polypeptide.

In some embodiments of the method of measuring a molecular flux rate or fractional synthesis rate of a protein or polypeptide, the method is carried out wherein the stable-isotope labeled precursor exposed sample is from a subject exposed to a stable-isotope precursor molecule selected from ²H₂O, ¹³C-labeled amino acid, ¹⁵N-labeled amino acid, and ¹⁷O-labeled amino acid. In some embodiments, the stable-isotope labeled precursor exposed sample is from a subject exposed to ²H₂O. In some embodiments, the first isotopologue is a ²H-isotopologue and the second isotopologue is a ¹³C-isotopologue.

In some embodiments of the method of measuring a molecular flux rate or fractional synthesis rate of a protein or polypeptide, the method is carried out wherein the mass isotopomer is an M1 mass isotopomer. In some embodiments, the mass isotopomer is an M1 mass isotopomer, and the abundance of the M1 mass isotopomer in the exposed sample differs from the control sample by less than 10%, less than 5%, or even less than 1%. In some embodiments of the method, the mass isotopomer of an immonium ion fragment derived from the protein or polypeptide is part of a mass isotopomer envelope, and the change in relative abundance of the first isotopologue between the exposed and control samples is less than 1% of the change in relative abundance of the total mass isotopomer envelope between the exposed and control samples. T In some embodiments of the method, the protein or polypeptide in the stable-isotope labeled precursor exposed sample has a stable-isotope enrichment of less than 1.0%, less than 0.5%, or even less than 0.1%.

In some embodiments of the method of measuring a molecular flux rate or fractional synthesis rate of a protein or polypeptide, the method is carried out wherein the exact masses of the first and second isotopologues differ by 9 or fewer millidaltons, 5 or fewer millidaltons, or even 3 or fewer millidaltons. In some embodiments, the method is carried out wherein the first isotopologue peak height and the second isotopologue peak height differ by less than 50%. In some embodiments, the method is carried out wherein the high-resolution mass spectrometric measurement is performed using an FT-ICR mass spectrometer.

In another aspect, the present disclosure provides a method of measuring a molecular flux rate or fractional synthesis rate of a protein or polypeptide comprising: (a) performing a high resolution mass spectrometric measurement of a stable-isotope labeled precursor exposed sample and a control sample, wherein each sample comprises the protein or polypeptide; (b) determining from the measurement of each sample the relative abundances of a first and a second isotopologue in each of a plurality of immonium ion fragments derived from the polypeptide, wherein the first and second isotopologues have different exact masses and are part of the same mass isotopomer of one of the plurality of immonium ion fragments; and (c) calculating the molecular flux rate of the polypeptide based on the relative abundances of the first isotopologue to the second isotopologue from the plurality of immonium ion fragments measured in the exposed and control samples.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

FIG. 1 shows a characteristic plateaued ²H₂O body water enrichment in a human subject of approximately 0.75% (p) and an average or effective p of 0.5% resulting from a standard low-dose ²H₂O-labeling protocol.

FIG. 2 shows a theoretical fractional synthesis rate of plasma haptoglobin in a human subject over time obtained from a standard low-dose ²H₂O-labeling protocol.

FIG. 3 shows the modeled relative abundances of the ¹³C and the ²H isotopologues of the M1-mass isotopomer in an immonium fragment ion of serine from a haptoglobin peptide that would be obtained after a standard low-dose ²H₂O-labeling protocol. In a sample from a subject with an average body water ²H₂O exposure of 0.50% over a period of 12 hours (see FIG. 1), the fraction of newly synthesized haptoglobin molecules will be ˜15% (see FIG. 2). This will result in a change in the ²H-isotopologue % relative abundance from a very low natural abundance value of ˜0.1% to ˜20% (lower ²H-trace). Relative abundance is the ratio of the abundance of the ²H-isotopologue of the M1-mass isotopomer to the sum of (i) the abundance of the ²H-isotopologue of the M1-mass isotopomer and (ii) the abundance of the ¹³C-isotopologue of the M1-mass isotopomer, or expressed algebraically ²H/(²H+¹³C). The theoretical maximal % relative abundance for the ²H-isotopologue of the M1-mass isotopomer under these labeling conditions, representing the value for this fragment ion of serine from this peptide in newly synthesized molecules of haptoblobin, is ˜70% of the abundance of the ¹³C-isotopologue (upper ²H trace). This ˜50% change in the ²H-isotopologue peak in the M1-mass isotopomer after 12 hours of label exposure provides a much higher signal than the <1% change in the M1-mass isotopomer envelope peak that is observable under these conditions (not shown).

FIGS. 4A & 4B illustrates a modeled high resolution mass spectrum of a sample polypeptide AAAEVNQEYGLDPK which has M1 mass isotopomer peak with a nominal mass of 1504 daltons, a chemical formula of C₆₅H₁₀₁N₁₇O₂₄, and a maximum of 34 potential deuterium incorporation sites (FIG. 4A). Within the envelope of the M1 mass isotopomer peak is a set of four isotopologue peaks due to isotopic species containing one of the following isotopes ¹⁵N, ¹³C, ¹⁷O, or ²H (FIG. 4B). Although part of the same nominal mass M1 mass isotopomer, these four isotopologues have four distinct exact masses separated by a total of 9 millidaltons.

FIG. 5 shows the modeled isotopomer and isotopologue abundance profiles for a sample peptide labeled under conditions where body water was at 1.0% enrichment with ²H₂O. The upper plot show the abundance profiles for the M0, M1, M2, and M3 isotopomers. The two lower plots show the modeled isotopologue abundance profiles within each of the M1 and M2 isotopomers. Large changes in relative abundances between unlabeled and labeled species are observed for the ²H-isotopologues of the M1 and M2 mass isotopomers.

FIG. 6 shows a modeled graph of the fractional synthesis (f) versus the relative abundance of the ²H to the ¹³C-isotopologue in the M1-mass isotopomer of the serine immonium ion. The relative abundances change substantially as a function of the fraction of newly synthesized protein molecules present (from 0 to 100% newly synthesized).

FIG. 7 shows the modeled relative abundances of the ²H to the ¹³C-isotopologues in the M1-mass isotopomer of the serine immonium ion as a function of the fraction of newly synthesized protein molecules (f) at different body water ²H₂O exposures (p). Even at relatively low values of fractional synthesis (f), such as 20%, the change in the relative abundance of the ²H to the ¹³C-isotopologue in the M1-mass isotopomer of the immonium ion of serine, is substantial, resulting in a large analytic signal for measurement of (f).

FIG. 8 shows modeled data and results that illustrate the use of more than one immonium ion fragment from a single peptide to provide internal analytic replicates that can improve analytic performance when calculating flux rates of a protein molecule. In this example, the peptide is SEPIPETNEGPVK and the immonium ions from serine (Ser) and glutamic acid (Glu) were analyzed by FT-ICR mass spectrometry. The upper figures show abundances of the peaks for the ²H and ¹³C-isotopologues in the M1-mass isotopomer of Ser and Glu immonium ions (the ¹³C isototopologue on the left and the ²H isotopologue on the right for each trace, with mass separation of 3 millidaltons). Comparable peak heights can be seen to be present for the 2 isotopologues of each immonium ion. The table in the lower left shows how the %²H@max calculation is different for each amino acid's immonium ion, based on its chemical formula and likelihood of having ²H metabolic incorporation from body ²H₂O. The text on the lower right shows the equations used for calculation of %²H and fractional synthesis (f) and the values of f measured in this example for Ser and Glu immonium ions. Thus, the mean value of f calculated from these two immonium ions has the advantage of being based on independent analytic replicates for the calculation.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Described herein are methods for measuring a molecular flux rate based on analysis of isotopologue abundance within a mass isotopomer, e.g., using a high resolution mass spectrometric measurement. These methods are based at least in part on the discovery described herein that metabolically labeling and analyzing relative abundances of isotopologues in selected mass isotopomers (e.g., through use of high mass-resolution mass spectrometry) may allow for the calculation of molecular flux rates with greater accuracy and/or precision than standard techniques. This is thought to be particularly advantageous for applications in which observed changes in mass isotopomer abundances are relatively modest, as with ²H₂O labeling protocols that are optimal for medical diagnosis and testing.

In certain aspects, provided herein are methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of a relative abundance of a first isotopologue from said enriched or isolated one or more stable isotope-labeled target molecules of interest, wherein the first and the second isotopologues are part of the same mass isotopomer and have different masses; (e) comparing the relative abundance of the first isotopologue to a control relative abundance of the first isotopologue, wherein the control relative abundance is a ratio of abundance of the first isotopologue from the one or more target molecules of interest before or without administration of the stable isotope-labeled precursor molecule; and (f) calculating a fraction of newly synthesized target molecules of interest based on the comparison of the relative abundance of the first isotopologue and the control relative abundance of the first isotopologue.

As used here, relative abundance is defined as the abundance, peak height, peak area or other parameter known in the art for expressing the quantitative abundance of a first molecular species compared to the quantitative abundance, peak height, peak area or other parameter known in the art of a second or more than one other molecular species, as measured by a mass spectrometer. Relative abundances, as defined here, can be expressed by any of several mathematical equations, including but not limited to the relative abundance of the first isotopologue expressed a ratio of the abundance of the first isotopologue to a sum of (i) the abundance of the first isotopologue and (ii) an abundance of a second isotopologue from said enriched or isolated one or more stable isotope-labeled target molecules of interest; or wherein the relative abundance of the first isotopologue is a ratio of abundance of the first isotopologue to the abundance of a second isotopologue from said enriched or isolated one or more stable isotope-labeled target molecules of interest; or wherein the relative abundance of the first isotopologue is a ratio of abundance of the first isotopologue to a sum of (i) the abundance of the first isotopologue and (ii) an abundance of a second and other isotopologues from said enriched or isolated one or more stable isotope-labeled target molecules of interest. In other aspects, provided herein are methods for measuring a molecular flux rate based on analysis of isotopologue abundance within a mass isotopomer, comprising: (a) administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; (b) obtaining from the subject a biological sample comprising the one or more stable isotope-labeled target molecules of interest; (c) enriching or isolating the one or more stable isotope-labeled target molecules of interest from said biological sample; (d) performing a high resolution mass spectrometric measurement of a relative abundance of a first isotopologue from said enriched or isolated one or more stable isotope-labeled target molecules of interest, wherein the relative abundance of the first isotopologue is a ratio of abundance of the first isotopologue to a sum of (i) the abundance of the first isotopologue and (ii) an abundance of a second isotopologue from said enriched or isolated one or more stable isotope-labeled target molecules of interest, wherein the first and the second isotopologues are part of the same mass isotopomer and have different masses; (e) comparing the relative abundance of the first isotopologue to a control relative abundance of the first isotopologue, wherein the control relative abundance is a ratio of abundance of the first isotopologue from the one or more target molecules of interest before or without administration of the stable isotope-labeled precursor molecule to a sum of (i) the abundance of the first isotopologue before or without administration of the stable isotope-labeled precursor molecule and (ii) an abundance of the second isotopologue from the one or more target molecules of interest without administration of the stable isotope-labeled precursor molecule; and (f) calculating a rate of breakdown or degradation of the target molecules of interest based on the comparison of the relative abundance of the first isotopologue and the control relative abundance of the first isotopologue.

### I. General Techniques

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) 3. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

U.S. Pat. No. 8,129,335 provides methods and disclosures that may be useful for practice of methods described herein.

### II. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Kinetic parameters” and “molecular flux rates” may be used interchangeably herein and may refer to the rate of synthesis, breakdown, and/or transport of a macromolecule (e.g., a polypeptide or polynucleotide). “Kinetic parameters” or “molecular flux rates” also refer to a macromolecule's input into or removal from a pool of macromolecules, and are therefore synonymous with the flow into and out of the pool of macromolecules.

“Exact mass” refers to the mass obtained by summing the masses of all the isotopes in the formula of a molecule (e.g., for CH₃NHD: exact mass=¹²C mass+¹⁴N mass+(4×¹H mass)+²H mass=32.04847).

“Nominal mass” refers to the mass obtained by rounding the exact mass of a molecule to the nearest integer value (e.g., for CH₃NHD: nominal mass=32).

“Isotopologues” refer to two or more molecular species that have identical elemental compositions but different isotopic compositions and different exact masses (e.g., CH₃NHD, ¹³CH₃NH₂, and CH₃¹⁵NH₂). Each isotopologue has a unique exact mass but may not have a unique structure. An isotopologue usually includes a family of isotopic isomers (isotopomers) which differ by the location of the isotopes on the molecule (e.g., CH₃NHD and CH₂DNH₂ are different isotopomers but the same isotopologue because they have the same isotopic composition and therefore the same exact mass). For example, ethanol, with a molecular formula of ¹²C₂ ¹H₆ ¹⁶O₁ for the monoisotopic M0 isotopomer, has 3 possible M1 isotopologues contributing to the M1-mass isotopomer (which is nominally 1 dalton heavier than the M0 mass isotopomer); these have isotopic elemental chemical formulas of ¹³C₁ ¹²C₁ ¹H₆ ¹⁶O₁, ¹²C₂ ¹H₆ ¹⁷O₁, and ¹²C₂ ²H₁ ¹H₅ ¹⁶O.

“Mass isotopomer” refers to family of isotopic isomers that have the same nominal mass, rather than isotopic composition or exact mass. A mass isotopomer may comprise different isotopologues (e.g., CH₃NHD, ¹³CH₃NH₂, CH₃¹⁵NH₂ are part of the same mass isotopomer but are different isotopologues). For example, the isotopologues CH₃NHD, ¹³CH₃NH₂, and CH₃¹⁵NH₂ all have the same nominal mass, and hence are part of the same mass isotopomer, even though each has a different exact mass. Each mass isotopomer is typically composed of more than one isotopologue and therefore has more than one exact mass associated with it. The mass isotopomer lowest in mass is represented as M0; for most organic molecules, the M0 species contains all ¹²C, ¹H, ¹⁶O, ¹⁴N, etc. Other mass isotopomers are distinguished by their mass differences from M0 (e.g., M1, M2, etc.). For a given mass isotopomer, the location or position of isotopes within the molecule is not specified and may vary (i.e., “positional isotopomers” are not distinguished).

“Mass isotopomer envelope” refers to the set of mass isotopomers associated with a molecule or ion fragment.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonymously with the term “mass isotopomer pattern.”

“Monoisotopic mass” refers to the exact mass of the molecular species that contains only the most abundant isotopes, e.g., only ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m₀, and the masses of other mass isotopomers are identified by their mass differences from m₀ (m₁, m₂, etc.).

A “high resolution mass spectrometric measurement” and terms related thereto may refer to a mass spectrometry measurement that is capable of distinguishing and quantifying different isotopologues within the same mass isotopomer. For example, a high resolution mass spectrometric measurement may distinguish one or more mass differences among ¹⁵N, ¹³C, ¹⁷O, and/or ²H-labeled isotopologues in the same mass isotopomer. To use the M1-mass isotopomer of ethanol as a specific example, a high resolution mass spectrometric measurement may resolve three different ethanol isotopologues having the isotopic compositions of ¹³C₁ ¹²C₁ ¹H₆ ¹⁶O₁, ¹²C₂ ¹H₆ ¹⁷O₁, and ¹²C₂ ²H₁ ¹H₅ ¹⁶O₁.

“Isotope-labeled water” includes water labeled with one or more specific heavy isotopes of either hydrogen or oxygen. Specific examples of isotope-labeled water include ²H₂O, ³H₂O, and H₂¹⁸O.

“Stable isotope-labeled precursor molecules” refer to a metabolite precursor that contains a stable isotope of an element that differs from the most abundant isotope of the element present in nature or cells, tissues, or organisms. Stable isotopic labels include specific heavy isotopes of elements, present in biomolecules, such as ²H, ¹³C, ¹⁵N, ¹⁷O, and ¹⁸O. Isotope labeled organic metabolite precursors include but are not limited to ²H₂O, ¹⁵NH₃, ¹³CO₂, H¹³CO₃, H₂¹⁷O, H₂¹⁸O, ²H-labeled amino acids, ¹³C-labeled amino acids, ¹³C-labeled polypeptides, ¹³C-labeled organic metabolites, ¹³C-labeled inorganic carbon compounds, ¹⁵N-labeled amino acids, ¹⁵N-labeled polypeptides, ¹⁵N-labeled inorganic nitrogenous compounds, ¹⁷O-labeled amino acids, ¹⁸O-labeled amino acids.

“Enriching” refers to methods of removing one or more components of a mixture of other similar compounds. As used herein, the term “partially purifying” may be used interchangeably.

“Isolating” refers to separating one compound from a mixture of compounds. For example, “isolating a macromolecule” refers to separating one specific macromolecule from all other macromolecules in a mixture of one or more macromolecules.

A “biological sample” encompasses any sample obtained from a cell, tissue, or organism. The definition encompasses blood and other liquid samples of biological origin, that are accessible from an organism through sampling by minimally invasive or non-invasive approaches (e.g., urine collection, blood drawing, needle aspiration, and other procedures involving minimal risk, discomfort or effort), as well as portions of solid tissue that may be obtained, e.g., using a surgical biopsy; surgical removal; percutaneous, endoscopic, transvascular, radiographic-guided or other non-surgical biopsy; euthanizing an experimental animal and removing tissue; collecting ex vivo experimental preparations; removing tissue at post-mortem examination; or other methods of collecting tissues. The definition also includes samples that have been manipulated in any way after their procurement, such as by treatment with reagents, solubilization, or enrichment for certain components, such as proteins or organic metabolites. The term “biological sample” also encompasses a clinical sample such as serum, plasma, other biological fluid, or tissue samples, and also includes cells in culture, cell supernatants and cell lysates. A volume of a body fluid may be used to refer to a liquid biological sample.

“Isotopically perturbed” refers to the state of an element or molecule that results from the explicit incorporation of an element or molecule with a distribution of isotopes that differs from the distribution found in nature, whether a naturally less abundant isotope is present in excess (enriched) or in deficit (depleted).

“Monomer” refers to a chemical unit that combines during the synthesis of a polymer and which is present two or more times in the polymer.

“Polymer” refers to a molecule synthesized from and containing two or more repeats of a monomer.

“Protein” (the term “polypeptide” may be used interchangeably herein) refers to a polymer of amino acids. As used herein, a “protein” may refer to long amino acid polymers as well as short polymers such as peptides.

“Polynucleotide” refers to a polymer of nucleotides. As used herein, a “polynucleotide” may refer to polymers such as DNA, RNA, cDNA, and so forth, and is meant to include polymers of naturally occurring polynucleotides and polymers of modified or unnatural polynucleotides, as well as polymers having a mixture or combination thereof.

### III. Methods of the Disclosure

The present disclosure is directed to methods measuring one or more molecular flux rate(s) based on analysis of isotopologue relative abundance within a mass isotopomer. The methods may include administering a stable isotope-labeled precursor molecule to a subject for a period of time sufficient for said stable isotope-labeled precursor molecule to enter into a biosynthetic precursor pool and label one or more target molecules of interest to produce one or more stable isotope-labeled target molecules of interest; obtaining from the subject a biological sample comprising the one or more stable isotope-labeled target molecules of interest; enriching or isolating the one or more stable isotope-labeled target molecules of interest from said biological sample; performing a high resolution mass spectrometric measurement of a relative abundance of a first isotopologue from said enriched or isolated one or more stable isotope-labeled target molecules of interest, wherein the relative abundance of the first isotopologue is a ratio of abundance of the first isotopologue to a sum of (i) the abundance of the first isotopologue and (ii) an abundance of a second isotopologue from said enriched or isolated one or more stable isotope-labeled target molecules of interest, wherein the first and the second isotopologues are part of the same mass isotopomer and have different exact masses; and (e) comparing the relative abundance of the first isotopologue to a control relative abundance of the first isotopologue, wherein the control relative abundance is a ratio of abundance of the first isotopologue from the one or more target molecules of interest before or without administration of the stable isotope-labeled precursor molecule to a sum of (i) the abundance of the first isotopologue before or without administration of the stable isotope-labeled precursor molecule and (ii) an abundance of the second isotopologue from the one or more target molecules of interest without administration of the stable isotope-labeled precursor molecule.

In some embodiments, comparing the relative abundance of the first isotopologue to the control relative abundance of the first isotopologue may be used to calculate a fraction of newly synthesized target molecules of interest, e.g., based on the change in relative abundance of the first and the second isotopologues in the enriched or isolated stable isotope-labeled target molecules of interest collected during or after administering the stable isotope-labeled precursor molecule to the subject, as compared to the relative abundance of the first and the second isotopologues in the target molecules of interest before or without administering the stable isotope-labeled precursor molecule. In some embodiments, this fraction of newly synthesized target molecules of interest may subsequently be used, inter alia, to calculate a replacement rate of the target molecules of interest.

In other embodiments, comparing the relative abundance of the first isotopologue to the control relative abundance of the first isotopologue may be used to calculate a rate of breakdown or degradation of the target molecules of interest, e.g., based on the rate of decrease over time in the relative abundance of the first and the second isotopologues in the enriched or isolated stable isotope-labeled target molecules of interest in the enriched or isolated stable isotope-labeled target molecules of interest collected during the period after administering the stable isotope-labeled precursor molecule to the subject.

A. Administering a Stable Isotope-Labeled Precursor Molecule

1. Labeled Precursor Molecules

a. Isotope Labels

The first step in measuring molecular flux rates involves administering an isotope-labeled precursor molecule to a cell, tissue, or organism. In some embodiments, the isotope labeled precursor molecule may be a stable isotope. Isotope labels that can be used include, but are not limited to, ²H, ¹³C, ¹⁵N, ¹⁷O, ¹⁸O, or other stable isotopes of elements present in organic systems. In one embodiment, the isotope label is ²H.

b. Precursor Molecules

The precursor molecule may be any molecule having an isotope label that is incorporated into an organic metabolite, such as a polypeptide or polynucleotide. Isotope labels may be used to modify all precursor molecules disclosed herein to form isotope-labeled precursor molecules.

In some embodiments, the entire precursor molecule may be incorporated into one or more organic metabolites. Alternatively, a portion of the precursor molecule may be incorporated into one or more organic metabolites.

Precursor molecules may include, but are not limited to, CO₂, NH₃, glucose, lactate, ²H₂O, acetate, and fatty acids.

i. Protein or Polypeptide Precursors

A protein or polypeptide precursor molecule may be any protein or polypeptide precursor molecule known in the art. These precursor molecules may be CO₂, NH₃, glucose, lactate, H₂O, acetate, and fatty acids.

Precursor molecules of proteins or polypeptides may also include one or more amino acids. The precursor may be any amino acid. The precursor molecule may be a singly or multiply deuterated amino acid. For example, the precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted or diluted with non-labeled amino acids. All isotope labeled precursors may be purchased commercially, for example, from Cambridge Isotope Labs (Andover, Mass.).

Protein precursor molecules may also include any precursor for post-translational or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non-essential amino acid is known—e.g. 4 in alanine, 2 in glycine, etc.

The precursor molecule for proteins may be water. The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—i.e., that the protein is newly synthesized. Analytically, the amino add derivative used must contain all the C—H bonds but must remove all potentially contaminating N—H and O—H bonds.

Hydrogen atoms from body water may be incorporated into free amino acids. ²H or ³H from labeled water can enter into free amino adds in the cell through the reactions of intermediary metabolism, but ²H cannot enter into amino acids that are present in peptide bonds or that are bound to transfer RNA. Free essential amino acids may incorporate a single hydrogen atom from body water into the .alpha.-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutrate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histine, the hydroxyl group in hydroxyproline or hydroxylysine, and others. Other amino adds synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from ¹⁸O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after post-translational modification step (e.g. methylation, hydroxylation, phosphorylation, prenylation, sulfation, carboxylation, acetylation or other known post-translational modifications).

Protein precursors for that are suitable for administration into a subject include, but are not limited to H₂O, CO₂, NH₃ and HCO₃, in addition to the standard amino acids found in proteins.

ii. Water as a Precursor Molecule

In one embodiment, isotope-labeled water may serve as a precursor in the methods described herein. Water is a precursor of proteins, polynucleotides, and many other organic metabolites. As such, labeled water may serve as a precursor in the methods taught herein. H₂O availability is probably never limiting for biosynthetic reactions in a cell (because H₂O represents close to 70% of the content of cells, or >35 Molar concentration), but hydrogen and oxygen atoms from H₂O contribute stochiometrically to many reactions involved in biosynthetic pathways: e.g.: R—CO—CH2-COOH+NADPH+H₂O→R—CH2CH2COOH (fatty acid synthesis).

As a consequence, isotope labels provided in the form of H- or O-isotope-labeled water are incorporated into biological molecules as part of synthetic pathways. Hydrogen incorporation can occur in two ways: into labile positions in a molecule (i.e., rapidly exchangeable, not requiring enzyme catalyzed reactions) or into stable positions (i.e., not rapidly exchangeable, requiring enzyme catalysis). Oxygen incorporation occurs in stable positions.

Some of the hydrogen-incorporating steps from cellular water into C—H bonds in biological molecules only occur during well-defined enzyme-catalyzed steps in the biosynthetic reaction sequence, and are not labile (exchangeable with solvent water in the tissue) once present in the mature end-product molecules. For example, the C—H bonds on glucose are not exchangeable in solution. In contrast, each of the following C—H positions exchanges with body water during reversal of specific enzymatic reactions: C-1 and C-6, in the oxaloacetate/succinate sequence in the Krebs' cycle and in the lactate/pyruvate reaction; C-2, in the glucose-6-phosphate/fructose-6-phosphate reaction; C-3 and C-4, in the glyceraldehyde-3-phosphate/dihydroxyacetone-phosphate reaction; C-5, in the 3-phosphoglycerate/glyceraldehyde-3-phosphate and glucose-6-phosphate/fructose-6-phosphate reactions.

Labeled hydrogen or oxygen atoms from water that are covalently incorporated into specific non-labile positions of a molecule thereby reveals the molecule's “biosynthetic history”—i.e., label incorporation signifies that the molecule was synthesized during the period that isotope-labeled water was present in cellular water.

The labile hydrogens (non-covalently associated or present in exchangeable covalent bonds) in these biological molecules do not reveal the molecule's biosynthetic history. Labile hydrogen atoms can be easily removed by incubation with unlabelled water (H₂O) (i.e., by reversal of the same non-enzymatic exchange reactions through which ²H or ³H was incorporated in the first place), however:

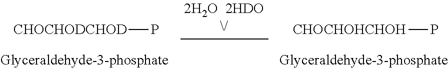

As a consequence, potentially contaminating hydrogen label that does not reflect biosynthetic history, but is incorporated via non-synthetic exchange reactions, can easily be removed in practice by incubation with natural abundance H₂O.

Isotope-labeled water may be readily obtained commercially. “Isotope-labeled water” or “heavy water” includes water labeled with one or more specific heavy isotopes of either hydrogen or oxygen. Specific examples of isotope-labeled water include ²H₂O, ³H₂O, and H₂¹⁸O. For example, ²H₂O may be purchased from Cambridge Isotope Labs (Andover, Mass.), and ³H₂O may be purchased from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 litres water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the present disclosure. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. As long as rapid changes in body water enrichment are prevented (e.g., by initial administration in small, divided doses), high body water enrichments of ²H₂O can be maintained with no toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

iii. Modes of Administering Precursors

Modes of administering the one or more isotope-labeled precursor molecules may vary, depending upon the absorptive properties of the isotope-labeled precursor and the specific biosynthetic pool into which each compound is targeted. Precursors may be administered to organisms, plants and animals including humans directly for in vivo analysis. In addition, precursors may be administered in vitro to living cells. Specific types of living cells include hepatocytes, adipocytes, myocytes, fibroblasts, neurons, pancreatic β-cells, intestinal epithelial cells, leukocytes, lymphocytes, erythrocytes, microbial cells and any other cell-type that can be maintained alive and functional in vitro.

Generally, an appropriate mode of administration is one that produces a steady state level of precursor within the biosynthetic pool and/or in a reservoir supplying such a pool for at least a transient period of time. Intravenous or oral routes of administration are commonly used to administer such precursors to organisms, including humans. Other routes of administration, such as subcutaneous or intra-muscular administration, optionally when used in conjunction with slow release precursor compositions, are also appropriate. Compositions for injection are generally prepared in sterile pharmaceutical excipients. Modes of administration may comprise continuous administration or discontinuous administration (e.g., a pulse chase).

In some embodiments, the one or more isotope-labeled precursor molecules may be administered to a human. In other embodiments, the one or more isotope-labeled precursor molecules may be administered to a veterinary or research subject, including without limitation mammals such as rodents, primates, hamsters, guinea pigs, horses, dogs, or pigs.

B. Obtaining a Biological Sample

A plurality of molecules of interest may be acquired from the cell, tissue, or organism. The one or more biological samples may be obtained, for example, by blood draw, urine collection, biopsy, or other methods known in the art. The one or more biological sample may be one or more biological fluids. The molecule of interest may also be obtained from specific organs or tissues, such as muscle, liver, adrenal tissue, prostate tissue, endometrial tissue, blood, skin, and breast tissue. Molecules of interest may be obtained from a specific group of cells, such as tumor cells or fibroblast cells. Molecules of interest also may be obtained, and optionally partially purified or isolated, from the biological sample using standard biochemical methods known in the art.

A sample may include a tissue histology specimen from tissues such as, for example, the gut, skin, organs, breast, prostate, brain, bone, muscle, liver, and gut. The sample may also be obtained from bodily fluids including, for example, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, and intestinal secretions. The sample may further include biofilms, microbiomes and other microbial organisms. The sample may be a clinical sample, upon which a clinical decision, diagnosis or prognosis can be made using the output generated according to the methods described herein.

The sample may be obtained, for example, by blood draw, urine collection, biopsy, or other methods known in the art. In some embodiments, the sample is obtained by taking a surgical biopsy; surgical removal of a tissue or portion of a tissue; performing a percutaneous, endoscopic, transvascular, radiographic-guided or other non-surgical biopsy; euthanizing an experimental animal and removing tissue; collecting ex vivo experimental preparations; removing tissue at post-mortem examination; or other methods known in the art for collecting tissue samples. The methods of obtaining a sample may also vary and be specific to the molecules of interest.

Standard techniques for preparing a sample for mass spectrometry include, for example, freezing and slicing, lyophilization, cryopreservation, ethanol dehydration, OCL preservation, and other suitable methods known in the art. In some embodiments, the samples are prepared on a slide with a coated surface that permits or increases energy-dependent volatilization of molecules from the surface of the slide.

The frequency of biological sampling can vary depending on different factors. Such factors include, but are not limited to, the nature of the molecules of interest, ease and safety of sampling, synthesis and breakdown/removal rates of the molecules of interest, and the half-life of a compound (chemical entity, biological factor, already-approved drug, drug candidate, drug lead, etc.).

C. Enriching or Isolating a Stable Isotope-Labeled Molecule of Interest

In some embodiments, a stable isotope-labeled target molecule of interest is enriched or isolated from a biological sample. Proteins, polypeptides, or other organic metabolites may be partially purified, enriched, or isolated, from a biological sample using standard biochemical methods known in the art. For example, suitable methods of enriching or isolating a protein may include, but are not limited to, immunoprecipitation, chromatography (e.g., by size exclusion, hydrophobic interaction, affinity, metal binding, immunoaffinity, or HPLC), centrifugation through a density gradient, etc. Suitable methods for enrichment and isolation may depend upon, for example, abundance of the molecule of interest, biochemical properties of the molecule of interest, the type of sample, and the relative degree of enrichment or purity required.

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the molecules of interest. The molecules of interest also may be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

In some embodiments, the one or more target molecules of interest may be proteins, peptides or amino acids isolated or derived from one or more polypeptides. In certain embodiments, the isotopologues (e.g., a first and a second isotopologue of the present disclosure) on which the high resolution mass spectrometric measurement is performed are derived from a fragment ion, the fragment ion being derived from the one or more stable isotope-labeled target molecules of interest.

In some embodiments, the target molecules of interest are proteins or polypeptides and the high resolution mass spectrometric measurement is performed on an immonium ion fragment derived from the protein or polypeptide. In some embodiments, the immonium ion fragments are derived from amino acid residues of the protein or polypeptide. Because the protein or polypeptide includes multiple different amino acid residues, it is possible to carry out replicate measurements of different immonium ion fragments from the same protein or polypeptide sample. Accordingly, in some embodiment, the methods of the present disclosure can be carried out wherein a high resolution mass spectrometric measurement is performed on a plurality of immonium ion fragments derived from a protein or polypeptide, wherein the plurality of fragments correspond to different amino acid residues from the protein or polypeptide.

In some embodiments, the one or more target molecules of interest are isolated from a cell, e.g., using immunoprecipitation, chromatography (e.g., by size exclusion, hydrophobic interaction, affinity, metal binding, immunoaffinity, or HPLC), centrifugation through a density gradient, and so forth.

D. Performing a High Resolution Mass Spectrometric Measurement

Isotopic enrichment in proteins, polypeptides, or other organic metabolites can be determined by various mass spectrometric methods. Mass spectrometers convert molecules such as proteins, polypeptides, or other organic metabolites into rapidly moving gaseous ions and separate them on the basis of their mass-to-charge ratios. The distributions of isotopes or isotopologues of ions, or ion fragments (e.g., immonium ion fragments), may thus be used to measure the isotopic enrichment in a plurality of proteins, polypeptides, or other organic metabolites. Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers. Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization. Different ionization methods are also known in the art. One key advance has been the development of techniques for ionization of large, non-volatile macromolecules including proteins and polynucleotides. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, two or more mass analyzers may be coupled (MS/MS) first to separate precursor ions, then to separate and measure gas phase fragment ions. These instruments generate an initial series of ionic fragments of a protein, and then generate secondary fragments of the initial ions. The resulting overlapping sequences allows complete sequencing of the protein, by piecing together overlaying “pieces of the puzzle” based on a single mass spectrometric analysis within a few minutes (plus computer analysis time).

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS (or other mass spectrometric modalities that analyze ions of proteins and organic metabolites, rather than small inorganic gases) is used to measure mass isotopomer abundances of organic molecules, hydrogen-labeled isotope incorporation from isotope-labeled water is amplified 3 to 7-fold, depending on the number of hydrogen atoms incorporated into the organic molecule from isotope-labeled water in vivo.

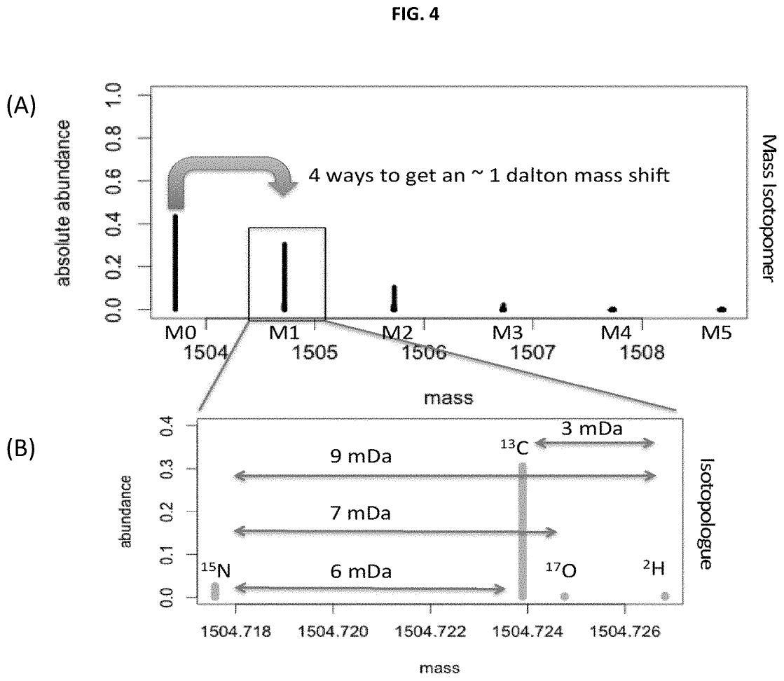

Certain aspects of the present disclosure relate to high resolution mass spectrometric measurements. As described herein and of particular relevance to kinetic measurements based on relative abundances of isotopologues, high resolution mass spectrometric measurements allow the resolution of one or more isotopologues within the same mass isotopomer, such as ¹⁵N, ¹³C, ¹⁷O, ¹⁸O, and/or ²H-labeled isotopologues in the same mass isotopomer (e.g., an M1 mass isotopomer). In some embodiments, a high resolution mass spectrometric measurement may refer to a measurement performed by a high resolution mass spectrometer capable of quantifying isotopologues that differ in mass by about 9 or fewer mDa, about 8 or fewer mDa, about 7 or fewer mDa, about 6 or fewer mDa, about 5 or fewer mDa, about 4 or fewer mDa, about 3 or fewer mDa, about 2 or fewer mDa, or about 1 or fewer mDa. In some embodiments, a high resolution mass spectrometric measurement may refer to a measurement performed by a high resolution mass spectrometer capable of quantifying isotopologues that differ in exact mass but share the same nominal mass. In some embodiments, a high resolution mass spectrometric measurement may refer to a measurement performed by a high resolution mass spectrometer capable of quantifying and resolving one or more isotopologues as depicted in FIGS. 4A and 4B.

In some embodiments, the high resolution mass spectrometer is a Fourier transform-based mass spectrometer, including without limitation a Fourier transform ion cyclotron resonance (FT-ICR) mass spectrometer and an Orbitrap mass spectrometer. In FT-ICR, as is well described in the art, ions are trapped in a Penning trap and excited at resonant cyclotron frequencies through circular orbits dictated by the Lorentz forces generated between a magnetic field and an orthogonal electric field. In Orbitrap, a barrel-like outer electrode at ground potential and a spindle-like central electrode are used to trap ions in trajectories rotating elliptically around the central electrode with oscillations along the central axis, confined by the balance of centrifugal and electrostatic forces. The use of both instruments employs a Fourier transform operation to convert a time domain signal (e.g., frequency) from detection of image current into a high resolution mass measurement. Further descriptions and details may be found, e.g., in Scheltema, R. A. et al. (2014) Mol. Cell Proteomics 13:3698-3708; Perry, R. H. et al. (2008) Mass. Spectrom. Rev. 27:661-699; and Scigelova, M. et al. (2011) Mol. Cell Proteomics 10:M111.009431.

In general, in order to determine a control isotopologue frequency distribution for a molecule of interest (e.g., a control relative abundance), a sample is taken before exposure of the sample to an isotopically labeled precursor. Such a measurement is one means of establishing in the cell, tissue or organism, the naturally occurring frequency of isotopologues of the molecule. When a cell, tissue or organism is part of a population of subjects having similar environmental histories, a population isotopologue frequency distribution may be used for such a background measurement. Additionally, such a control isotopologue frequency distribution may be estimated, using known average natural abundances of isotopes. For example, in nature, the natural abundance of ¹³C present in organic carbon in 1.11%. Methods of determining such isotopologue frequency distributions are discussed below. Typically, samples of the molecule of interest are taken prior to and following administration of an stable isotopically labeled precursor molecule to the subject and analyzed for isotopologue frequency as described below.

E. Comparing Isotopologue Relative Abundances

The high resolution mass spectrometric measurement techniques described above are used to measure relative abundances of isotopologues, e.g., within the same mass isotopomer or within different mass isotopomers. In some embodiments, relative abundance may refer to relative peak height on a mass spectrum. In some embodiments, relative abundance may refer to area under the peak. In some embodiments, relative abundance may be expressed as a ratio toward the parent (all ¹²C) mass isotopomer, as described below. It is appreciated that any calculation means which provides relative and absolute values for the abundances of isotopologues, in a sample may be used in describing such data, for the purposes of the present disclosure.

In some embodiments, relative abundances of two or more isotopologues may be measured. Any pair of isotopologues, including ²H—, ¹³C-, ¹⁵N-, and ¹⁷O-isotopologues may be measured and used in calculations described herein. In certain embodiments, the relative abundances of a ²H-isotopologue and a ¹³C-isotopologue may be measured. In certain embodiments, the relative abundances of a ²H-isotopologue and a ¹³C-isotopologue in the same mass isotopomer may be measured.

In some embodiments, the two or more isotopologues each has a distinct exact mass and is part of the same mass isotopomer, e.g., having different exact masses of a mass difference resolvable by high resolution mass spectrometry. In some embodiments, the two or more isotopologues represent two distinct exact masses in an M1 mass isotopomer. In certain embodiments, the relative abundances of a ²H-isotopologue and a ¹³C-isotopologue in an M1 mass isotopomer may be measured.

In some embodiments, relative abundance of an isotopologue of interest may be expressed as a ratio of the relative abundance of the isotopologue of interest (e.g., a ²H-labeled isotopologue from the M1 mass isotopomer) to the sum of the relative abundance of the isotopologue of interest and the relative abundance of a second isotopologue having a different exact mass in the same mass isotopomer (e.g., a ¹³C-labeled isotopologue from the M1 mass isotopomer). Without wishing to be bound to theory, it is thought that the large increase in signal intensity of, e.g., a ²H-labeled isotopologue relative to its naturally occurring form, coincident with the very small mass difference between the ¹³C-isotopologue and the ²H-isotopologue in the M1-mass isotopomer (2.9 millidaltons) after isotope labeling, favors the use of high resolution mass spectrometry, in which ions with highly similar masses are subject to fewer potential biases (e.g., different behavior of ions in the trap as a function of their mass) than those with more dissimilar masses.

In other embodiments, high resolution mass spectrometric measurement may be used to measure relative abundances of isotopologues in different mass isotopomers. Without wishing to be bound to theory, it is thought that such methods may yield less background signal, e.g., with a deuterium or ⁵N label, than in a mass isotopomer (which includes naturally abundant ¹³C). For example, a leucine with 3 deuterium labels (d3-leucine) may be administered, and a relative abundance of a 3×²H-isotopologue of the M3 mass isotopomer may be measured and compared to a ¹³C isotopologue in the M0 isotopomer, instead of measuring the M3 and M0 mass isotopomers by traditional lower resolution mass spectrometric measurements. It should be noted that there is in theory or practice little or no analytic benefit from measuring the abundance of an isotopologue rather than the abundance of a mass isotopomer when the isotopologue is essentially the only isotopic species present in the mass isotopomer. In such instances, quantitation of the isotopologue and the mass isotopomer are essentially identical. In other applications, however, particularly with administration of heavy water, it is thought that comparing isotopologues in the M2 or M3 mass isotopomers may be more complicated than staying within the M1 mass isotopomer—the higher mass isotopomers contain combinations of ²H, ¹³C, and other isotopologues, which “smears out” their capacity to be separated.

In some embodiments, the relative abundances of a first isotopologue and a second isotopologue are of comparable peak heights or signal intensities. Without wishing to be bound to theory, it is thought that this may reduce the potential analytic impact of non-linearities of detector quantitation or differences in ion counting statistics when quantifying isotopologue relative abundances. In some embodiments, “comparable” peak heights or signal intensities may refer to peak heights or signal intensities that differ from each other by less than about 50%, less than about 25%, or less than about 10%.

In some embodiments, e.g., for labeling and measuring turnover rates of proteins, stable isotope labeling using 1% ¹⁵N results in an maximum ¹⁵N:¹³C isotopologue abundance ratio of 1:5 while 5% ¹⁵N pool enrichment results in a maximum ¹⁵N: ¹³C isotopologue abundance ratio of approximately 1.3:1, with a mass difference of 6 mDa between these two isotopologues.

In some embodiments, the methods described herein allow the high resolution mass spectrometric measurement of a change in the relative abundances of two or more isotopologues from the same mass isotopomer even if the abundance of the mass isotopomer as a whole changes only a slight amount or not at all. This may be the case when distinct isotopologues are entering and leaving a mass isotopomer simultaneously, which may occur with significant molecular flux rates even if the overall mass isotopomer abundance does not change. For example, ²H-isotopologues may enter an M1 mass isotopomer while ¹³C-isotopologues are leaving, resulting in significant molecular flux rates with only a slight change, or no change, in the relative abundance of the M1 mass isotopomer itself. Advantageously, the methods of the present disclosure allow for ways to measure these molecular flux rates, which would be invisible to techniques that only measure mass isotopomer abundances. In some embodiments, a small or no change in relative abundance of a mass isotopomer may refer to a change in theoretical maximal relative abundance that is less than about 5% of the total mass isotopomer envelope measured or to a change in measurable or observable relative abundance that is less than about 1% of the total mass isotopomer envelope measured.

Quantification of molecular flux rates or kinetics by measuring abundance ratios of isotopologues, particularly in the same mass isotopomer, rather than comparisons elsewhere in the entire isotopomer envelope, constitutes a novel analytical approach for measuring flux, one which has the potential to enable greater quantitative accuracy. The ¹³C and ²H isotopologues of the M1-mass isotopomer can be differentiated and quantified by mass spectrometry to provide information about molecular flux rates of ethanol in a biological system, after introduction of ¹³C or ²H-labeled ethanol in vivo. The relationship between the isotopomer and isotopomer peaks is illustrated by FIG. 4, which shows modeled isotopomer and isotopologue mass spectra for the peptide sequence AAAEVNQEYGLDPK. This peptide has a chemical formula of C₆₅H₁₀₁N₁₇O₂₄ and a maximum of 34 potential deuterium incorporation sites. The monoisotopic mass isotopomer of the peptide (denoted M0) has only one isotopologue; it includes 1 molecular species with a unique isotopic elemental chemical formula and mass (see in FIG. 4A). As shown in FIG. 4B, the model peptide sequence for the M1 mass isotopomer cluster consists of carbon, hydrogen, nitrogen and oxygen atoms and has 4 unique elemental isotope chemical formulas or isotopologues (unique elemental formulas resulting in a nominal 1 dalton mass shift). As illustrated by the range of mDa differences in peak masses shown in FIG. 4B, quantification of changes in relative abundance of isomeric isotopologues within the M1 mass isotopomer require an analytical measurement where the quantified analytes differ in mass by 3 to 9 thousandths of a Dalton (millidalton [mDa]), depending on which isotopologues are quantified.

The natural abundance of the stable isotope of carbon, ¹³C, is ˜1.1%, which leads to an appreciable ¹³C isotopologue contribution to the M1 isotopomer signal of carbon-containing molecules, such as proteins or polypeptides. The ¹³C isotopologue of a typical biomolecule of these types is the most abundant molecular species in the M1 or higher mass isotopomer clusters, prior to stable isotope enrichment.

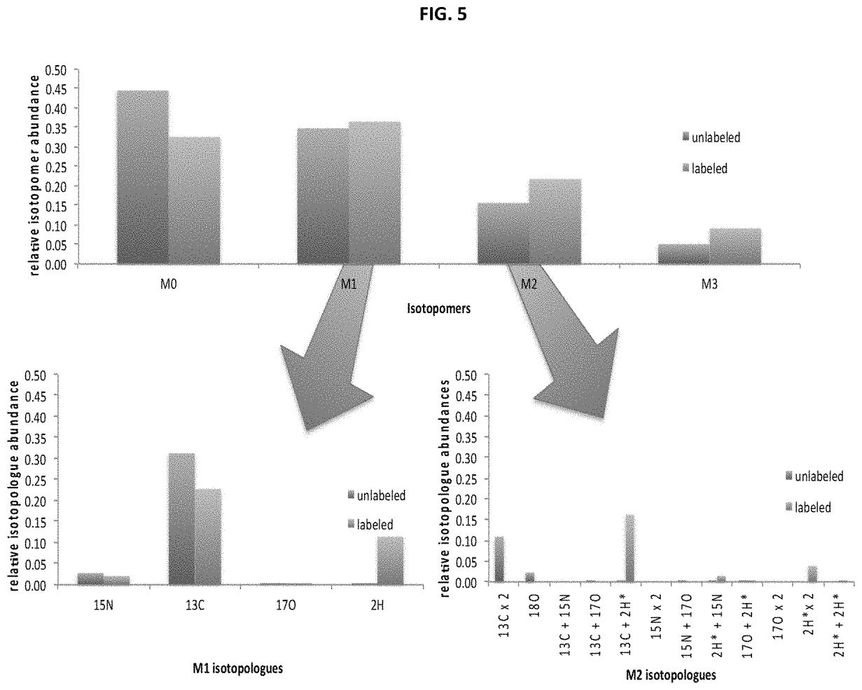

In some embodiments, e.g., wherein turnover or synthesis rates of proteins are measured by use of high resolution mass spectrometry, deuterated water (²H₂O, heavy water) may be used as the source of stable isotope label in vivo, and deuterium body water enrichment may typically be in the range of 1-2% in human subjects. For a peptide from a protein that is analyzed by mass spectrometry under these labeling conditions, with a typical deuterium enrichment in body water of 1%, the maximal change in the fractional abundance of the M0 mass isotopomer is ˜11.9%, or a loss of 26% of the natural abundance M0 signal intensity, as shown in FIG. 5. Accurately measuring a loss of 26% in the M0 signal requires accurately quantifying abundances of the M1 and other mass isotopomers in the cluster for this peptide, typically M0-M3. The M1 mass isotopomer, for example, will exhibit a change in fractional abundance of +1.7%, These mass isotopomers differ by ˜1 dalton each in the +1 charge state of the molecular ion. As illustrated in FIG. 5, the isotopomer cluster is therefore spread over ˜4 daltons with a wide range of relative abundances. This spread in the m/z dimension increases the likelihood of interfering analytic features or ionic contaminants relative to the more narrow mass range and ion abundances present in the isotopologue cluster within a single mass isotopomer.

For a typical deuterium enrichment in body water of 1%, the maximal change in relative abundance of the ²H-isotopologue compared to the ¹³C-isotopologue in the M1 isotopomer is +11.0%, representing an increase of 3,217% over the naturally occurring isotopologue abundance of the ²H-isotopologue compared to the ¹³C-isotopologue in the M1 isotopomer (FIG. 5). This large increase in signal intensity, coincident with the very small mass difference between the ¹³C-isotopologue and the ²H-isotopologue in the M1-mass isotopomer (2.9 millidaltons) after isotope labeling, favors better analytical accuracy and precision in quantitation of relative abundances, in comparison to quantitation of mass isotopomer baundances. In addition, the likelihood of contamination by ions derived from a complex matrix (multiplexing) is obviously less over a narrow mass range such as 3 millidaltons, compared to a broad mass range such as 3 daltons.

Quantifying normalized changes in isotopologue abundance requires accurately quantifying at least two of the isotopologues in the M1-mass isotopomer cluster. It is beneficial to quantify the ¹³C and ²H isotopologue abundances, as there is only a 3 mDa mass difference (see, e.g., FIG. 4B), given that mass differences contribute to errors in measured isotope abundance in FT-ICR mass analyzers (Perry, R. H. et al. (2008) Mass. Spectrom. Rev. 27:661-699; and Scheltema, R. A. et al. (2014) Mol. Cell Proteomics 13:3698-3708). Accordingly, theoretical accuracy for measuring the relative abundances of isotopologues within a mass isotopomer cluster may be enhanced if the relative signal intensities of the isotopologues are of comparable magnitude and the mass differences are small. To calculate the protein fractional synthesis, one can model peptide isotopologue abundances based on the precursor pool (e.g., ²H₂O) isotope enrichment and the number of stable isotope incorporation sites in each analyte (for each peptide, in this example). Based on the theoretical maximum isotopologue abundance for each peptide due to metabolic stable isotope labeling over a labeling time interval, one can then determine the fractional synthesis rate or % new protein made by comparing the isotopologue abundances measured to theoretical values for 100% and 0% new protein (Hellerstein, M. K. and Neese, R. A. (1992) Am. J. Physiol. 263: E988-1001; Hellerstein, M. K. and Neese, R. A. (1999) Am. J. Physiol. 276: E1146-1170; Hellerstein, M. K. et al. (1991) J. Clin. Invest. 87:1841-1852; Schwarz, J. M. et al. (1995) J. Clin. Invest. 96:2735-2743; Strawford, A. et al. (2004) Am. J. Physiol. Endocrinol. Metab. 286: E577-588; Neese, R. A. et al. (1995) J. Biol. Chem. 270:14452-14466; Hellerstein, M. K. et al. (1997) Am. J. Physiol. 272: E163-172; Hellerstein, M. K. et al. (1997) J. Clin. Invest. 100:1305-1309; Louie, K. B. et al. (2013) Sci. Rep. 3:1656; Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; Price, J. C. et al. (2012) Mol. Cell Proteomics 11:1801-1814; Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057).

F. Calculations

In some embodiments, based on measurements of isotopologue relative abundances as described above, measured excess molar ratios may be calculated for isolated isotopologue species of a molecule. The practitioner then compares measured internal pattern of excess ratios to the theoretical patterns. Such theoretical patterns can be calculated using the binomial or multinomial distribution relationships as described in U.S. Pat. Nos. 5,338,686; 5,910,403; and 6,010,846. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. Pat. No. 7,001,587. In addition to the above-cited references, calculation software implementing the method is publicly available from Professor Marc Hellerstein, University of California, Berkeley.

Relative abundance is then calculated. By way of example, the relative abundance of a ²H-isotopologue compared to the ¹³C isotopologue in the M1-mass isotopomer of a molecule can be calculated from their measured peak heights or areas by the equation, ²H/(²H+¹³C), where ²H refers to the measured peak height or area of the ²H isotopologue and ¹³C refers to the measured peak height or area of the ¹³C isotopologue in the M1-mass isotopomer.

Relative abundance of an isotopologue of interest may then be compared to a control relative abundance. In some embodiments, a control relative abundance may refer to a relative abundance of an isotopologue as it naturally occurs, e.g., without administration of an exogenous isotope label. In some embodiments, a control relative abundance may refer to a relative abundance of the same type of target molecule of interest from which the experimental isotopologue was derived but taken from a sample collected prior to administration of the stable isotope-labeled precursor molecule. In some embodiments, a control relative abundance may refer to a relative abundance of the same type of target molecule of interest from which the experimental isotopologue was derived but taken from a sample collected without administration of the stable isotope-labeled precursor molecule. In some embodiments, a control relative abundance may refer to a known, calculated, or expected relative abundance based on the natural abundance of an isotope or isotopes within a particular isotopologue.

As discussed above, in some embodiments, relative abundance of an isotopologue may be expressed as a ratio of the isotopologue abundance to the sum of the isotopologue abundance and the abundance of a different isotopologue, such as (without limitation) a distinct isotopologue in the same mass isotopomer. To use the ²H- and ¹³C-isotopologues of an M1 mass isotopomer as an example, relative abundance of the ²H-isotopologue may be expressed as:

$$Relative abundance {}^{2} HM 1 = \frac{Abundance {}^{2} HM 1}{( Abundance {}^{2} HM 1 + Abundance {}^{13} CM 1 )}$$

In some embodiments, comparing the relative abundance of an isotopologue to a corresponding control relative abundance may refer to a determination of enrichment or depletion (excess relative abundance, which may be a positive or negative number, respectively) during or after administration of the stable isotope-labeled precursor, e.g.,

$${( Realtive {abundance}_{x} )}_{e} - {( Relative {abundance}_{x} )}_{b} = {( \frac{Abundance {}^{2} HM 1}{( Abundance {}^{2} HM 1 + Abundance {}^{13} CM 1 )} )}_{e} - {( \frac{Abundance {}^{2} HM 1}{( Abundance {}^{2} HM 1 + Abundance {}^{13} CM 1 )} )}_{b} ,$$

where subscript e refers to enriched and b refers to baseline or natural abundance.

In order to determine the fraction of stable isotope-labeled molecules of interest that were actually newly synthesized during a period of precursor administration, the measured excess molar ratio (EM_{X}) is compared to the calculated theoretical maximum enrichment value, A_{X}*, which describes the enrichment in 100% newly synthesized stable-isotope labeled molecules of interest for an isotopologue, to reveal the isotopologue excess ratio which would be expected to be present if all isotopologues were from newly synthesized molecules assembled during the period of exposure to label (maximal Δ relative abundance). In some embodiments, a method of determining rate of synthesis includes calculating the proportion of mass isotopically labeled subunits present in the precursor pool, and using this proportion to calculate an expected frequency of molecules containing different numbers of mass isotopically labeled subunits. These expected frequencies (maximal Δ relative abundances) are then compared to the actual, experimentally determined isotopologue frequencies (observed Δ relative abundances). From these values, the proportion or fraction of the molecules of interest which were synthesized during a selected label incorporation period can be determined. Thus, the rate of synthesis, calculated as fraction or absolute mass of new molecules synthesized per unit of time, during such a time period is also determined. In another embodiment, a fully replaced (100% labeled) reference molecule is isolated from a subject and used as an internal standard to calculate the fractional synthesis of a molecule of interest over a time period. The excess relative abundance of an isotopologue in the fully replaced reference molecule (maximal Δ relative abundance) is used as the denominator and the excess relative abundance of an isotopologue in the molecule (observed Δ relative abundance) of interest is used as the numerator in the equation to calculate the proportion or fraction of the molecules of interest which were synthesized during a selected label incorporation period

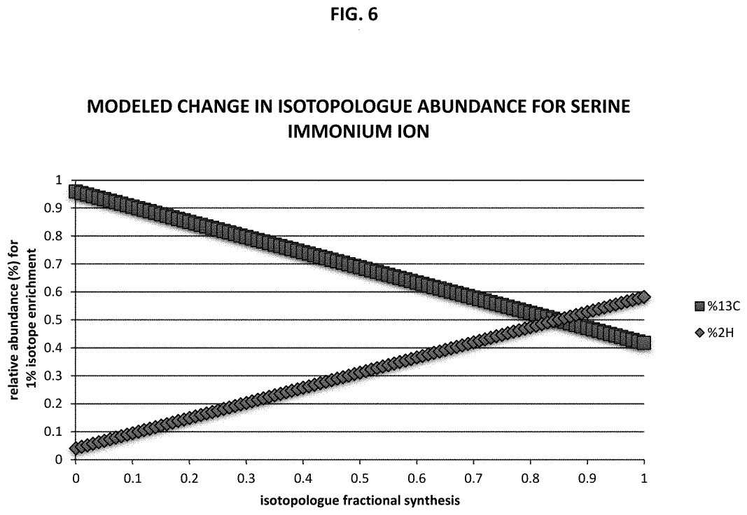

In these embodiments, the fraction of newly synthesized target molecules of interest may be calculated based on equations known in the art which comprise a comparison of the relative abundance of an isotopologue and a corresponding control relative abundance of the isotopologue in a molecule of interest to a maximal relative abundance of the isotopologue in a fully replaced reference molecule. The relationship of fractional synthesis to changes in relative abundance of ²H and ¹³C isotopologues of a serine immonium ion is illustrated in FIG. 6. As shown by the plot of FIG. 6, the relative abundances change substantially as a function of the fraction of newly synthesized protein molecules present (from 0 to 100% newly synthesized).

Equations for determining a fraction of newly synthesized target molecules of interest during or after a period of stable isotope label administration are well known in the art and include without limitation:

$${( Fraction newly synthesized )}_{i} = \frac{{( observed Δ Relative {abundance}_{x} )}_{i}}{{( maximal Δ Relative {abundance}_{x} )}_{i}}$$

where the maximal ΔRelative abundance for isotopologue x is calculated based on the labeling conditions present in a subject or sample during or after administration of a stable isotope-labeled precursor molecule for a period of time i.

In some embodiments, this calculation is derived from the measured or inferred isotopic abundance of the stable isotope-labeled precursor molecule from which the target molecules of interest were synthesized in the subject at the site of biosynthesis of the target molecules of interest, e.g., the calculated theoretical maximum enrichment value, A_{X}*, which describes the enrichment in 100% newly synthesized stable-isotope labeled molecules of interest for an isotopologue, using equations known in the art (see e.g., Hellerstein, M. K. and Neese, R. A. (1999) Am. J. Physiol. 276: E1146-1170); Busch, R. et al. (2007) Nat. Protoc. 2:3045-3057; Holmes W., Angel T., Li K., & Hellerstein M K. Dynamic Proteomics: In vivo proteome-wide measurement of protein kinetics using metabolic labeling. Methods Enz., In Press, 2015).

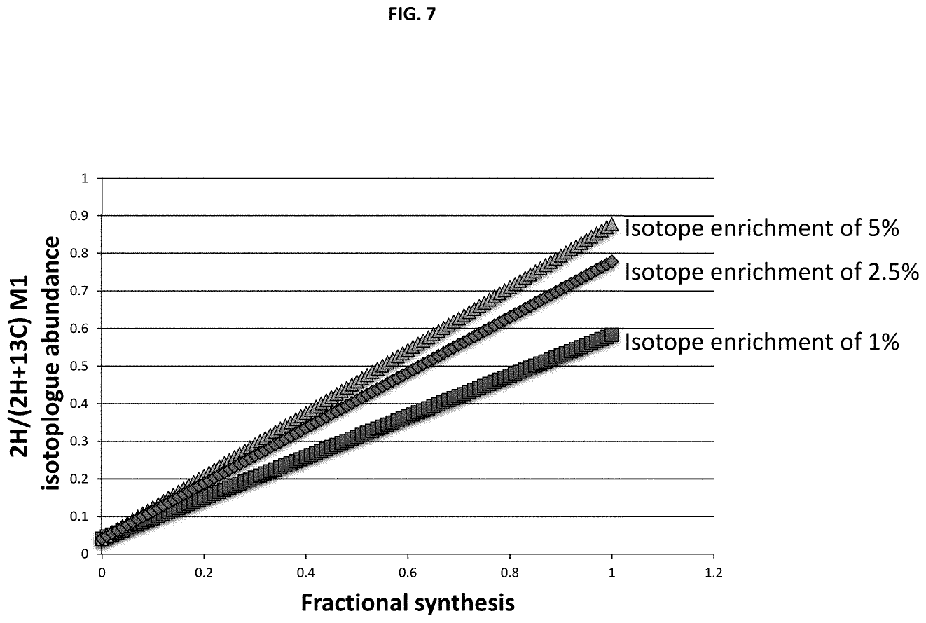

The use of immonium ion fragment isotopologue abundances is particularly advantageous for the measurement fractional synthesis (f) because the very large theoretical maximum enrichment value, A_{X}* for such isotopologues can provide a high signal even at low values of fractional synthesis (f). This advantage is illustrated in the plots of FIG. 7 which show modeled relative abundances of the ²H to the ¹³C-isotopologues in the M1-mass isotopomer of a serine immonium ion as a function of the fraction of newly synthesized protein molecules (f) at different body water ²H₂O exposures (p). Even at relatively low values of fractional synthesis (f), such as 20%, the change in the relative abundance of the ²H to the ¹³C-isotopologue in the M1-mass isotopomer of the immonium ion of serine, is substantial, resulting in a large analytic signal for measurement of (f).

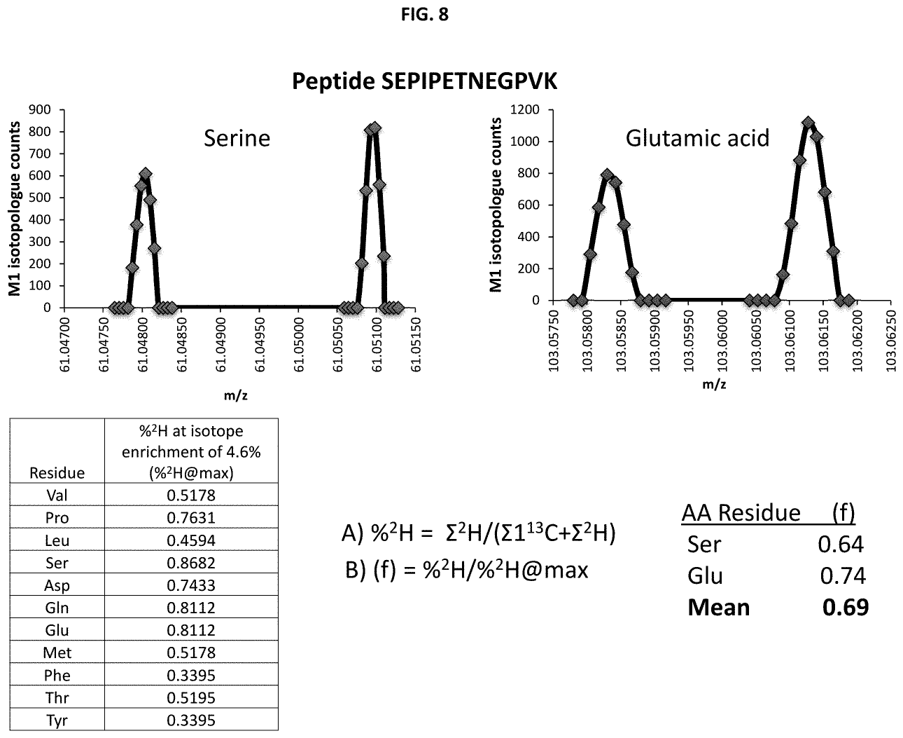

In some embodiments, the methods of measuring metabolic flux rates of the present disclosure can be carried out using measurements from a plurality of immonium ion fragments from different amino acids of a single peptide as internal analytic replicates for improving analytic performance when calculating flux rates of a protein molecule. Such methods are illustrated by the modeled data and results from an exemplary peptide SEPIPETNEGPVK as depicted in FIG. 8. In this example, the immonium ion fragments from serine (Ser) and glutamic acid (Glu) of the peptide, SEPIPETNEGPVK were analyzed by FT-ICR mass spectrometry. The upper figures show abundances of the peaks for the ²H and ¹³C-isotopologues in the M1-mass isotopomer of Ser and Glu immonium ions (the ¹³C isototopologue on the left and the ²H isotopologue on the right for each trace, with mass separation of 3 millidaltons). Comparable peak heights can be seen to be present for the 2 isotopologues of each immonium ion. As noted elsewhere herein, such may reduce the potential analytic impact of non-linearities of detector quantitation or differences in ion counting statistics when quantifying isotopologue relative abundances. The table in the lower left of FIG. 8 shows the %²H@max values calculated for each different amino acid immonium ion based on its chemical formula and likelihood of having ²H metabolic incorporation from body ²H₂O. The equations (A) and (B) were used for calculation of %²H and fractional synthesis (f), respectively. The values of (f) for Ser and Glu immonium ions were 0.64, and 0.74, respectively. The mean (f) value of 0.69 was then calculated from these two immonium ion (f) values, showing the potential advantage of having a plurality of independent analytic replicates available from a single sample measurement.

In some embodiments, precursor-product relationship may then be applied. For the continuous labeling method, the isotopic enrichment is compared to asymptotic (i.e., maximal possible) enrichment and kinetic parameters (e.g., synthesis or replacement rates) are calculated from precursor-product equations. The fractional synthesis rate (kₛ) may be determined by applying the continuous labeling, precursor-product formula:

kₛ=[−ln(1−f)]/t

where f=fraction newly synthesized=product enrichment/asymptotic precursor/enrichment (e.g., as described above); t=time of label administration of contacting in the system studied; and the units of replacement rate are fraction per unit of time.

In some embodiments, more than one biological sample may be obtained at different points following administration of a stable isotope-labeled precursor. In this way, a fraction of newly synthesized target molecules of interest may be calculated for the period between administration of the stable isotope-labeled precursor and obtaining the first sample, between administration of the stable isotope-labeled precursor and obtaining the second sample, and/or between obtaining the first sample and obtaining the second sample. In some embodiments, such calculations may be used to calculate observed fractional synthesis from the change in relative abundance of an isotopologue by comparison to the theoretical maximum change in relative abundance of an isotopologue at a particular percentage or level of enrichment.

In some embodiments, a rate of breakdown or degradation of the target molecules of interest may be calculated based on a comparison of the relative abundance of an isotopologue and the control relative abundance of the isotopologue, e.g., as described above. Equations for determining a rate of breakdown or degradation of the target molecules of interest during or after a period of stable isotope label administration are well known in the art. For example, in a discontinuous labeling method, the rate of decline in isotope enrichment is calculated and the kinetic parameters of the molecules of interest are calculated from exponential decay equations of the change in relative abundance of an isotopologue.

Breakdown rate constants (k_{d}) may be calculated based on an exponential or other kinetic decay curve. For an example of a ²H-isotopologue of an M1-mass isotopomer (²H-M1), calculation of k_{d} can be from the equation known in the art,

k_{d}=[−ln relative abundance ²HM1]/t

Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

In some embodiments, one or more of the calculations and/or methods described herein may be used as a diagnostic test. Without wishing to be bound to theory, it is thought that applying the methods described herein may improve the utility of any diagnostic test using a non-radioactive, stable isotopic tracer, and these methods may be particularly advantageous for application in heavy water (²H₂O) labeling protocols, e.g., when changes in mass isotopomer abundances are relatively modest (such as for low dose or brief duration ²H₂O labeling protocols). For example, ²H₂O labeling may be used to measure mass isotopomer abundances (MIAs) and mass isotopomer distributions (MIDs) in the kinetic analysis of lipids (Hellerstein, M. K. et al. (1991) J. Clin. Invest. 87:1841-1852; Schwarz, J. M. et al. (1995) J. Clin. Invest. 96:2735-2743; and Strawford, A. et al. (2004) Am. J. Physiol. Endocrinol. Metab. 286: E577-588), intermediary metabolites (Neese, R. A. et al. (1995) J. Biol. Chem. 270:14452-14466; Hellerstein, M. K. et al. (1997) Am. J. Physiol. 272: E163-172; Hellerstein, M. K. et al. (1997) J. Clin. Invest. 100:1305-1309; and Louie, K. B. et al. (2013) Sci. Rep. 3:1656), proteins (Papageorgopoulos, C. et al. (1999) Anal. Biochem. 267:1-16; Busch, R. et al. (2006) Biochim. Biophys. Acta. 1760:730-744; Price, J. C. et al. (2012) Anal. Biochem. 420:73-83; and Price, J. C. et al. (2012) Mol. Cell Proteomics 11:1801-1814) and cells (Macallan, D. C. et al. (1998) Proc. Natl. Acad. Sci. USA 95:708-713; Neese, R. A. et al. (2002) Proc. Natl. Acad. Sci. USA 99:15345-15350; Hellerstein, M. K. et al. (1999) Nat. Med. 5:83-89; and Busch, R. et al. (2007) Nat. Protoc. 2:3045-3057). Exemplary molecules of interest and pathological states for which the methods described herein may find use in diagnostic applications include without limitation those described in U.S. Pat. No. 8,005,623.

In some embodiments, a diagnostic test may be used in the diagnosis, management, or treatment selection of a human patient. In other embodiments, a diagnostic test may be used in the diagnosis, management, or treatment selection of a veterinary patient or research subject, including without limitation mammals such as rodents, primates, hamsters, guinea pigs, horses, dogs, or pigs. For example, in some embodiments, the molecular flux rates in one or more metabolic pathways of interest may contribute to the prognosis, survival, morbidity, mortality, stage, therapeutic response, symptomology, disability or other clinical factor of the disease of interest. In one embodiment, the molecular flux rates in the one or more metabolic pathways being measured may be relevant to an underlying molecular pathogenesis, or causation of, one or more diseases. In another embodiment, the molecular flux rates in one or more metabolic pathways of interest may contribute to the initiation, progression, severity, pathology, aggressiveness, grade, activity, disability, mortality, morbidity, disease sub-classification or other underlying pathogenic or pathologic feature of the disease of interest. In some embodiments, one can quantitate the molecular flux rates of one or more molecules of interest within one or more targeted metabolic pathways and use the information as a biomarker of medical diagnosis, prognosis, or therapeutic efficacy of drug or combination drug treatment.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

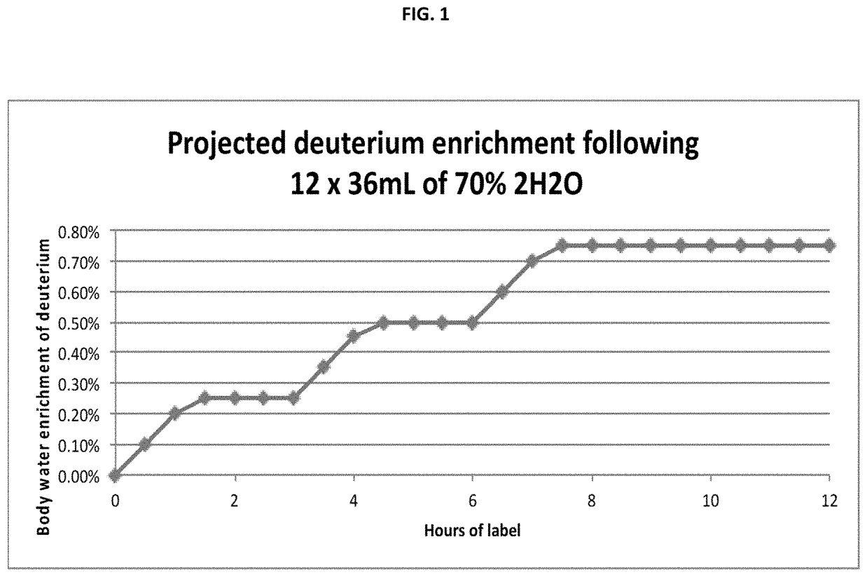

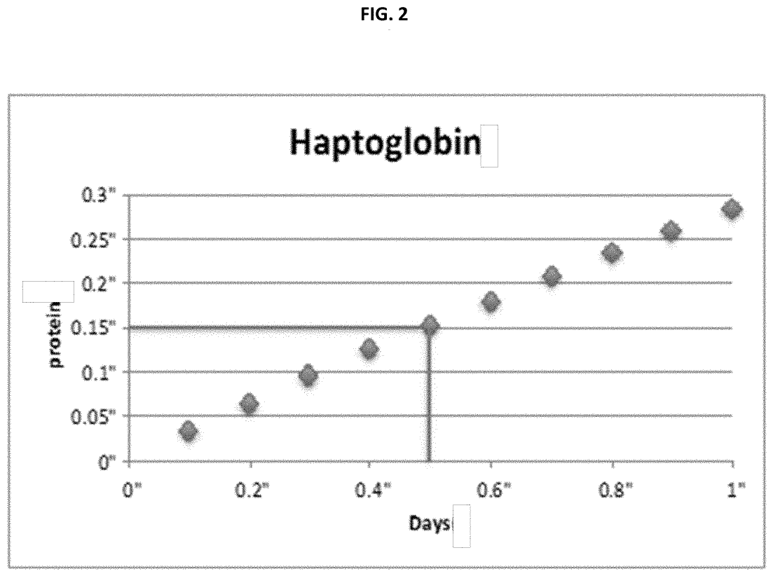

FIGS. 1 and 2 exemplify a standard low-dose ²H₂O-labeling protocol in which a healthy volunteer takes 4 drinks (50 ml @ 70% ²H₂O) of ²H₂O over the course of 12 hours. The exemplary target molecule of interest for molecular flux rate measurement is the protein, plasma haptoglobin, which is known to have a fractional replacement rate of approximately 33% per day. As shown in FIG. 1, this protocol results in a projected deuterium body water enrichment that plateaus at approximately 75% (p), with an average or effective (p) of 0.5%. FIG. 2 shows that, following one half day of water labeling with the protocol shown in FIG. 1, ˜15% of the haptoglobin molecules present will be newly synthesized.

For most tryptic peptides, the parent M0 mass isotopomer will exhibit the largest change in relative abundance (enrichment) under these labeling conditions. The M0 mass isotopomer of a prototypic haptoglobin polypeptide will have a peak theoretical label fractional incorporation (A*0) of ˜−0.038 (i.e., −3.8%), modeled at the average body water ²H₂O enrichment (p) of 0.50%. The expected change in enrichment of the M0 mass isotopomer (EM0) at 15% (f) will therefore be 0.038×0.15=0.0057, or 0.57% change in relative abundance or enrichment of the M0 mass isotopomer. The analytic uncertainty in EM0 on a Time of Flight mass spectrometer is ˜+/−0.50% EM0, so that the uncertainty in measured EM0 is +/−88% of the theoretical value (0.50%/0.57%). As discussed elsewhere herein, this large uncertainty in calculated fractional synthesis measurement is unlikely to be useful in medical practice, whereas calculations based on isotopologue abundances can provide much more useful data.

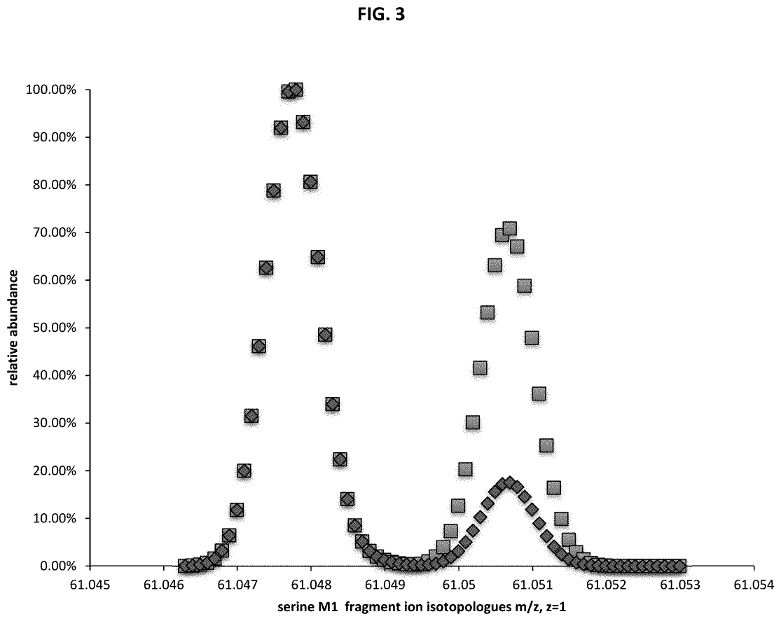

FIG. 3 shows the modeled relative abundances of the ¹³C and the ²H isotopologues of the M1-mass isotopomer in an immonium fragment ion of serine from a haptoglobin peptide that would be obtained from a subject with an average body water ²H₂O exposure of 0.50% over a period of 12 hours (see FIG. 1). The fraction of newly synthesized haptoglobin molecules will be ˜15% which results in a change in the ²H-isotopologue % relative abundance from a very low natural abundance value of ˜0.1% to ˜20% (lower ²H trace, FIG. 3). The relative abundance is the ratio of the abundance of the ²H-isotopologue in the M1-mass isotopomer to the sum of (i) the abundance of the ²H-isotopologue of the M1-mass isotopomer and (ii) the abundance of the ¹³C-isotopologue of the M1-mass isotopomer. The ratio can be expressed algebraically ²H(²H+¹³C). The theoretical maximal % relative abundance for the ²H-isotopologue of the M1-mass isotopomer under these labeling conditions, representing the value for this fragment ion of serine from this peptide in newly synthesized molecules of haptoblobin, is ˜70% of the abundance of the ¹³C-isotopologue (upper ²H trace, FIG. 3). Thus, this example illustrates the potential 50% change in the ²H-isotopologue peak in the M1-mass isotopomer after 12 hours of label exposure. Such a large change will provide a much higher signal than the <1% change in the M1-mass isotopomer envelope peak that is observable under these conditions (not shown).

The large 50% change in the ²H-isotopologue peak in the M1-mass isotopomer also highlights the potential quantify fractional synthesis with isotope enrichment levels much lower than described here. Moreover, at low values body water enrichment (e.g., p values below 0.5%), calculations indicate that the generation of the ²H-isotopologue in the M1-mass isotopomer can actually be favored over the generation of higher mass isotopomers in the molecule. Thus, in the 2H-isotopologue based methods disclosed herein, it may be favorable to use lower isotope enrichment levels because fewer combinations of ²H-label occur, which would shunt ²H-label into M2 and higher mass isotopomers. In this manner, 2H-isotopologue measurements in the M1-mass isotopomer can turn the clinical goal of reducing dose and duration of heavy water labeling into an analytic advantage. In principle, the measurement of isotopologues in the M1-mass isotopomer thereby substantially improves analytic sensitivity and reproducibility, compared to measurement of mass isotopomer abundances.

In addition, the use of isotopologue relative abundances for immonium ion fragments of peptides provides an additional and very important analytic advantage over the measurement of mass isotopomer abundances. Each amino acid in a peptide contains a unique immonium fragment that can give rise to an M1 mass isotopomer, and each M1 isotopomer includes isotopologues each with a distinct exact mass that can be measured using high resolution mass spectrometry. For example Table 2 below lists the calculated “natural abundance %²H” and “%²H@max” values for immonium ion fragments from a range of 15 different amino acids, along with the exact masses of these isotopologues in the M1 mass isotopomers.

TABLE 2

| | | % Natural | % Maximum | ¹³C | ²H |
|---|---|---|---|---|---|
| | | abundance ²H | labeled ²H at body | isotopologue | isotopologue |
| | Immonium | without body | ²H₂O isotope | in M1 mass | in M1 mass |
| Amino | ion chemical | ²H₂O isotope | enrichment of 0.5% | isotopomer | isotopomer |
| Acid | formula | enrichment | (% ²H@max) | exact mass | exact mass |
| Arg | C₅H₁₃N₄ | 3.47 | 23.79 | 130.1168 | 130.1197 |
| Asn | C₃H₇N₂O₁ | 3.18 | 22.66 | 88.0586 | 88.0616 |
| Asp | C₃H₆NO₂ | 2.73 | 22.66 | 89.0427 | 89.0456 |
| Glu | C₄H₈NO₂ | 3.03 | 31.24 | 103.0583 | 103.0612 |
| Gln | C₄H₉N₂O₁ | 2.70 | 31.24 | 102.0743 | 102.0772 |
| Iso/Leu | C₅H₁₂N | 3.18 | 8.34 | 87.0998 | 87.1027 |
| Lys | C₅H₁₃N₂ | 3.44 | 4.68 | 102.1107 | 102.1136 |
| Met | C₄H₁₀NS | 3.34 | 11.41 | 105.0562 | 105.0591 |
| Phe | C₈H₁₀N | 1.63 | 1.73 | 121.0841 | 121.0871 |
| Pro | C₄H₈N | 2.69 | 22.95 | 71.0685 | 71.0714 |
| Ser | C₂H₆NO₁ | 4.07 | 41.36 | 61.0477 | 61.0507 |
| Thr | C₃H₈NO₁ | 3.60 | 3.01 | 75.0634 | 75.0663 |
| Tyr | C₈H₁₀NO₁ | 1.63 | 2.26 | 137.0790 | 137.0820 |
| Val | C₄H₁₀N | 3.34 | 6.05 | 73.0841 | 73.0871 |

Typically, proteins and polypeptides that are target molecules of interest for isotopologue relative abundance analysis will include some or all of these amino acids that produce immonium ion fragments. Accordingly, in embodiments where a protein or polypeptide is the target molecule of interest, the method disclosed herein can be carried out wherein for a stable-isotope labeled sample the measurement of relative abundance of isotopologues in a mass isotopomer is repeated for a plurality of M1 isotopomers of immonium ion fragments produced by the protein or polypeptide of interest. This plurality of isotopologue relative abundance measurements for the various different immonium ion fragments can provide multiple non-redundant, independent measurements of isotopologue signals, and thereby provide more precise measurements of isotopologue relative abundances, and better values for the calculation of the metabolic flux rate or kinetic parameters of the target protein or polypeptide from which they were derived (see FIG. 8). Thus, this method of multiplexing of immonium ion measurements from a single peptide represents an analytic advantage for providing greater precision and accuracy.

The analytical performance of different immonium ion isotopologues may differ in accuracy and robustness for measuring protein or polypeptide fractional synthesis. Certain subsets of immonium ions may provide greater accuracy than others for measuring isotope enrichment and calculating fractional synthesis in the context of very low levels of isotope enrichment.

The high resolution mass spectrometric methods disclosed herein based on measurement of changes in isotopologue relative abundances in a single mass isotopomer rather than methods of measuring changes in total mass isotopomer relative abundances (MIAs and MIDs), provide the surprising advantage of greater accuracy and precision. Moreover, another surprising advantage is that the necessary high-resolution mass spectrometric measurements can be achieved using FT-ICR instruments, such as the Orbitrap, which are considered less accurate and reproducible at measuring relative abundances (of mass isotopomers) than ToF or quadrupole instruments. These unexpected advantages of the methods disclosed herein can be extended further by utilizing isotopologue relative abundance measurements from a plurality of immonium ion fragment isotopomers associated with a single peptide in a multiplex fashion. The method thereby can provide a plurality of independent replicate measurements on a single sample that improves the accuracy and precision in determining molecular flux rates and or fractional synthesis rates of proteins and polypeptides.

In a further exemplification of the methods disclosed herein, 5 normal human subjects can be treated using a standard labeling protocol where the healthy volunteer takes 4 drinks of deuterated water (50 ml @ 70% ²H₂O) a day for 3 days. This 3-day labeling treatment results in a plateaued deuterium body water enrichment of approximately 1% (p) and an average or effective p of 0.5%). Blood (plasma) is collected from the subjects at 6, 24, 48, and 72 hours after initiation of ²H₂O labeling. Body water enrichment is measured by GC-MS following established protocols (Voogt, Awada et al. 2007, Price, Holmes et al. 2012). The goal is to simplify and reduce operational requirements for measuring isotope enrichments, to allow less exposure to tracer, by 50-80% (i.e., to ˜20-50% of current labeling requirements), and reduce the metabolic labeling duration from >7 days to less than 2-days. Isotopologue abundance measurements as described herein can be made on samples from plasma of the subjects at early time points when deuterium enrichment is low. These experimental analyses can include multiple time-points; gel band-separations or antibody pull-downs at each time point, to reduce complexity of samples analyzed; triplicate measurements; and comparisons of different analytic approaches. Proteins with higher turnover rate constants (k) will have greater isotope incorporation and are models for very short duration labeling protocols. Conversely, proteins with lower values of k will have less isotope incorporation and will represent a higher bar for analytical performance.

The measurement of relative abundances of immonium ion fragment isotopologues within the same mass isotopomer can reveal changes in molecular flux rates of a protein or polypeptide under conditions where there is a small change or no change in the relative abundance of the same mass isotopomer. This situation often occurs for ion fragmentss of molecules that are of relatively high molecular weight (e.g, >1,000 daltons), where ²H-isotopologues enter and ¹³C-isotopologues leave said mass isotopomer due to molecular flux and the inflow and outflow rates roughly cancel each other out. In these instances, the larger changes in relative abundances of the ²H-isotopologue and the ¹³C-isotopologue can allow measurement of the molecular flux.

Throughout this application, various publications and patents have been referenced. The disclosures of these publications and patents in their entirety are hereby incorporated by reference into this application to more fully describe the state of the art to which this invention pertains.

While various specific embodiments have been illustrated and described, it will be appreciated that various changes can be made without departing from the spirit and scope of the invention(s).

What is claimed is:

1. A method for measuring a molecular flux rate or fractional synthesis rate of a protein or polypeptide based on analysis of isotopologue abundance within a mass isotopomer from an immonium ion fragment, comprising:
(a) administering a stable isotope-labeled precursor molecule to a subject for a period of time sufficient for said stable isotope-labeled precursor molecule to enter into a biosynthetic precursor pool and label one or more protein or polypeptide molecules of interest to produce one or more stable isotope-labeled target molecules of interest;
(b) obtaining from the subject a biological sample comprising the one or more stable isotope-labeled protein or polypeptide molecules of interest;
(c) enriching or isolating the one or more stable isotope-labeled protein or polypeptide molecules of interest from said biological sample;
(d) performing a high resolution mass spectrometric measurement of a relative abundance of a first isotopologue from said enriched or isolated one or more stable isotope-labeled protein or polypeptide molecules of interest, wherein the relative abundance of the first isotopologue is a ratio of abundance of the first isotopologue to a sum of (i) the abundance of the first isotopologue and (ii) an abundance of a second isotopologue from said enriched or isolated one or more stable isotope-labeled protein or polypeptide molecules of interest, in a preferred embodiment wherein the first and the second isotopologues have different exact masses and are part of the same mass isotopomer of an immonium ion fragment;
(e) comparing the relative abundance of the first isotopologue to a control relative abundance of the first isotopologue, wherein the control relative abundance is a ratio of abundance of the first isotopologue from the one or more protein or polypeptide molecules of interest before or without administration of the stable isotope-labeled precursor molecule to a sum of (i) the abundance of the first isotopologue before or without administration of the stable isotope-labeled precursor molecule and (ii) an abundance of the second isotopologue from the one or more protein or polypeptide molecules of interest without administration of the stable isotope-labeled precursor molecule; and
(f) calculating a fraction of newly synthesized protein or polypeptide molecules of interest based on the comparison of the relative abundance of the first isotopologue and the control relative abundance of the first isotopologue.

2. The method of claim 1, further comprising calculating a replacement rate of the protein or polypeptide molecules of interest based on the calculated fraction of newly synthesized protein or polypeptide molecules of interest.

3. The method of any one of claims 1-2, wherein the stable isotope-labeled precursor molecule is ²H₂O.

4. The method of claim 3, wherein the first isotopologue is a ²H-isotopologue, and wherein the second isotopologue is a ¹³C-isotopologue.

5. The method of any one of claims 1-2, wherein the stable isotope-labeled precursor molecule is selected from the group consisting of a ¹⁵N-labeled amino acid, a ¹⁵N-labeled polypeptide, and a ¹⁵N-labeled inorganic nitrogenous compound.

6. The method of any one of claims 1-2, wherein the stable isotope-labeled precursor molecule is selected from the group consisting of a ¹³C-labeled amino acid, a ¹³C-labeled polypeptide, a ¹³C-labeled organic metabolite, and a ¹³C-labeled inorganic carbon compound.

7. The method of any one of claims 1-2, wherein the stable isotope-labeled precursor molecule is ¹⁷O-labeled H₂O or ¹⁸O-labeled H₂O.

8. The method of any one of claims 1-7, wherein the mass isotopomer is an M1-mass isotopomer.

9. The method claim 1, wherein the production of molecular ions and isolation of fragment ions occurs on a high-resolution mass spectrometer operating at such resolving power that isotope label can be resolved on at least one fragment of the molecule, wherein the abundance of isotope label in the original molecular ions is established on the basis of abundance of the isotope label in said fragment as well as on probability of the particular isotope label to propagate from the molecular ion to the fragment ion.

10. The method of any one of claims 1-9, further comprising obtaining from the subject at least a second biological sample comprising the one or more stable isotope-labeled protein or polypeptide molecules of interest, wherein the first and second biological samples are obtained at different times, and wherein calculating the fraction of newly synthesized protein or polypeptide molecules of interest comprises calculating a fraction of protein or polypeptide molecules of interest synthesized before obtaining the first biological sample and a fraction of protein or polypeptide molecules of interest synthesized before obtaining the second biological sample.

11. The method of any one of claims 1-10, wherein the high resolution mass spectrometric measurement is performed using a high resolution mass spectrometer capable of quantifying isotopologues that differ in mass by nine or fewer millidaltons.

12. The method of claim 11, wherein the high resolution mass spectrometric measurement is performed using a high resolution mass spectrometer capable of quantifying isotopologues that differ in mass by three or fewer millidaltons.

13. The method of claim 11 or claim 12, wherein the high resolution mass spectrometer is an FT-ICR mass spectrometer.

14. The method of any one of claims 1-13, in a preferred embodiment wherein the relative abundances of the first isotopologue and the second isotopologue are of comparable peak heights or signal intensities (e.g., within 50% of each other).

15. The method of any one of claims 1-14, wherein the calculated fraction of newly synthesized protein or polypeptide molecules of interest, the replacement rate of the protein or polypeptide molecules of interest, the rate of breakdown or degradation of the protein or polypeptide molecules of interest, or any combination thereof is used in the diagnosis, management, or treatment selection of a human or veterinary patient.

16. The method of any one of claims 1-15, wherein the subject is a human.

17. A method of measuring a molecular flux rate or fractional synthesis rate of a protein or polypeptide comprising:
(a) performing a high resolution mass spectrometric measurement of a stable-isotope labeled precursor exposed sample and a control sample, wherein each sample comprises the protein or polypeptide;
(b) determining from the measurement of each sample the relative abundance of a first isotopologue to a second isotopologue, wherein the first and second isotopologues are part of the same mass isotopomer of an immonium ion fragment derived from the protein or polypeptide; and
(c) calculating the molecular flux rate or fractional synthesis rate of the protein or polypeptide based on the determined relative abundances of the first isotopologue to the second isotopologue for the exposed and control samples.

18. The method of claim 17, wherein step (b) is repeated to determine relative abundances for a first isotopologue to a second isotopologue in a plurality of different immonium ion fragments derived from the protein or polypeptide.

19. The method of claim 17 or 18, wherein the stable-isotope labeled precursor exposed sample is from a subject exposed to a stable-isotope precursor molecule selected from ²H₂O, ¹³C-labeled amino acid, ¹⁵N-labeled amino acid, and ¹⁷O-labeled amino acid.

20. The method of claim 17 or 18, wherein the stable-isotope labeled precursor exposed sample is from a subject exposed to ²H₂O.

21. The method of claim 17 or 18, wherein the first isotopologue is a ²H-isotopologue and the second isotopologue is a ¹³C-isotopologue.

22. The method of claim 17 or 18, wherein the mass isotopomer is an M1 mass isotopomer.

23. The method of claim 17 or 18, wherein the exact masses of the first and second isotopologues differ by 9 or fewer millidaltons, 5 or fewer millidaltons, or even 3 or fewer millidaltons.

24. The method of claim 17 or 18, wherein the first isotopologue peak height and the second isotopologue peak height differ by less than 50%.

25. The method of claim 17 or 18, wherein the high-resolution mass spectrometric measurement is performed using an FT-ICR mass spectrometer.

26. A method of measuring a molecular flux rate or fractional synthesis rate of a protein or polypeptide comprising:
(a) performing a high resolution mass spectrometric measurement of a stable-isotope labeled precursor exposed sample and a control sample, wherein each sample comprises the protein or polypeptide;
(b) determining from the measurement of each sample the relative abundances of a first and a second isotopologue in each of a plurality of immonium ion fragments derived from the polypeptide, wherein the first and second isotopologues have different exact masses and are part of the same mass isotopomer of one of the plurality of immonium ion fragments; and
(c) calculating the molecular flux rate of the polypeptide based on the relative abundances of the first isotopologue to the second isotopologue from the plurality of immonium ion fragments measured in the exposed and control samples.
